(19) United States
(12) Patent Application Publication
(10) Pub. No.: US 2017/0049881 A1
(43) Pub. Date: Feb. 23, 2017

(54) EPITOPES CROSS-REACTIVE BETWEEN HSV-1, HSV-2 AND VZV AND METHODS FOR USING SAME

(71) Applicant: UNIVERSITY OF WASHINGTON, SEATTLE, WA (US)

(72) Inventors: David M. KOELLE, SEATTLE, WA (US); Lichen JING, SEATTLE, WA (US); Kerry LAING, SEATTLE, WA (US); Christine JOHNSTON, SEATTLE, WA (US); Anna WALD, SEATTLE, WA (US)

(73) Assignee: UNIVERSITY OF WASHINGTON, SEATTLE, WA (US)

(21) Appl. No.: 15/308,076

(22) PCT Filed: May 1, 2015
(86) PCT No.: PCT/US15/28937

§ 371 Date: Oct. 31, 2016

Related U.S. Application Data
(60) Provisional application No. 61/987,985, filed on May 2, 2014.

(51) Int. Cl. A61K 39/245; C07K 14/005; C12N 7/00

(52) U.S. Cl. CPC A61K 39/245; C12N 7/00; C07K 14/005; C12N 2710/16634; A61K 2039/53

(57) ABSTRACT

The invention provides epitopes of HSV and VZV that are cross-reactive and are useful for the prevention and treatment of alphaherpesvirus infection. T-cells having specificity for antigens of the invention have demonstrated cytotoxic activity against cells loaded with virally-encoded peptide epitopes, and in many cases, against whole virus. The identification of immunogenic antigens responsible for T-cell specificity provides improved anti-viral therapeutic and prophylactic strategies. Compositions containing epitopes or polynucleotides encoding epitopes of the invention provide effectively targeted vaccines for prevention and treatment of alphaherpesvirus infection.

This application claims benefit of U.S. provisional patent application No. 61/987,985, filed May 2, 2014, the entire contents of which are incorporated by reference into this application.

### STATEMENT REGARDING FEDERALLY SPONSORED RESEARCH

This invention was made with government support under grant number AI094019 awarded by the National Institutes of Health. The government has certain rights in the invention.

### TECHNICAL HELD OF THE INVENTION

The invention relates to molecules, compositions and methods that can be used for the treatment and prevention of viral infection and other diseases. More particularly, the invention identifies epitopes of varicella zoster virus (VZV), herpes simplex virus type 1 (HSV-1), and herpes simplex virus type 2 (HSV-2) proteins that can be used for methods involving molecules and compositions having the antigenic specificity of VZV and HSV-specific T cells. In addition, the invention relates to methods for detecting, treating and preventing VZV and HSV infection, as well as methods for inducing an immune response to VZV and HSV. The epitopes described herein are also useful in the development of diagnostic and therapeutic agents for detecting, preventing and treating viral infection and other diseases.

### BACKGROUND OF THE INVENTION

VZV causes two main diseases in humans: chickenpox and shingles (herpes zoster). VZV is an alphaherpesvirus, like HSV-1 and HSV-2, and includes double-stranded DNA, has about 70 genes (open reading frames, or ORFs) and establishes latent infection in neurons. The HSV types 1 and 2 and VZV are evolutionarily related and have regions of identical or similar protein sequences in some proteins.

Viral sequences that are identical or closely related in HSV-1 and VZV that drive cross reactive T cell responses have been disclosed. Chiu et al., PLoS Pathog., March 2014; 10(3): e1004008, describes the HSV-1 UL40 184-192/VZV ORF18 epitope. This epitope has slight sequence variability between HSV-1 and VZV and a little more variation in HSV-2. T cells see all three of HSV-1, HSV-2 and VZV. Chiu et al. discusses the concept of a pan-herpesvirus vaccine. Chiu et al. finds that CD8 T cells in the blood that recognize a peptide epitope in VZV protein ORF18 can cross recognize a similar but not identical peptide epitope in HSV-1 protein UL40, and HSV-2 protein UL40. Chiu et al. does not show that the CD8 T cells in the blood can see the whole viruses, The HSV-1 version of this peptide, HSV-1 UL40 amino acids 184-192, had already been previously discovered to be a CD8 T cell epitope recognized by blood T-cells (Jing et al., 2012, J. Clin. Invest. 122(2):654-673).

Ouwendjik et al., J. Immunol., 2014 Apr. 15; 192(8):3730-9, found an epitope in HSV-1 and HSV-2 and VZV, a ten amino peptide epitope in the protein known as IE62 in VZV, amino acids 918-927, that is identical in sequence to a protein known as ICP4 in HSV-1 amino acids 999-1008 and identical in sequence of IPC4 protein from HSV-2 amino acids 1027-1036, which has identical sequence in each of HSV-1, HSV-2 and VZV. The gene for IE62 in VZV has another systemic number; ORF62 and ORF71. It occurs in two copies in the VZV genome as both open reading frame (ORF) 62 and 71. The ICP4 protein in HSV-1 and HSV-2 is encoded by a gene that is sometimes called ORF RS1 that also occurs as two copies in the HSV genome; in the case of HSV the two copies are not given different ORF names or numbers. The same T cell can potentially see all three viruses, Ouwdendjik et al. shows that CD4 T-cells that recognize this peptide can also recognize the whole viruses involved. Ouwendjik et al. shows that a peptide that is identical can elicit human T cells that cross react with HSV-1, HSV-2, and VZV.

Shingles is a reactivation of latent VZV. It causes vesicular (blister-like) rash, nerve pain, and typically affects a single dermatome. Pain can be prolonged and disabling, and quality of life is often reduced. There are about 1.5 to about 4.0 cases of shingles per 1000 per year, and up to about 1 million cases per year in the United States. About 10% to 30% of the population may be affected in their lifetime. The incidence of shingles increases with age, as does the severity of the disease, the associated complications, and the need for hospitalization. Shingles can be fatal, and the chance of death increases with age. As more than half of the cases are in people over the age of 60, the complications associated with VZV infection have a significant health care impact.

Herpes simplex type 1 (HSV-1) infects about 60% of people in the United States. Most people have either no symptoms or bothersome recurrent sores on the lips or face. Medically serious consequences of HSV-1 include herpes simplex encephalitis (HSE). HSE is usually a recurrence of HSV-1, and occurs in otherwise healthy, immunocompetent people. HSE can be fatal, and typically results in long term brain damage. Herpes simplex keratitis (HSK) is another serious consequence. HSK is part of a spectrum of HSV eye diseases that consume considerable health care resources; HSK can lead to blindness and a need for corneal transplantation. These and other complications are rare on a per-patient basis, but given the high prevalence of HSV-1, overall have a significant health care impact.

There is no vaccine for HSV and there is an imperfect VZV vaccine for chickenpox and shingles. The VZV vaccine contains a live attenuated vOka strain of VZV. The vaccine is given to children to prevent chicken pox, but is not safe in immune compromised children. The vaccine is also administered to adults to prevent shingles. However, the vaccine is not very effective or safe for immune compromised adults. There is a need for both safer and more effective VZV and HSV vaccine candidates.

### SUMMARY OF THE INVENTION

The invention provides compositions comprising VZV and HSV viral proteins termed epitopes, recognized by CD4 and CD8 T-cells that elicit cross-reactive immunity. In some aspects, the same immune cells can “see” both VZV and HSV, such as both HSV-1 and HSV-2. In other aspects, the immune cells can see VZV and HSV-1. In other aspects of the invention, the immune cells can only see VZV or HSV.

The invention provides VZV and HSV antigens, polypeptides comprising VZV and HSV antigens, polynucleotides encoding the polypeptides, vectors, and recombinant viruses containing the polynucleotides, antigen-presenting cells (APCs) presenting the polypeptides, immune cells directed against VZV and HSV, and pharmaceutical compositions. Compositions comprising these polypeptides, polynucleotides, viruses, APCs and immune cells can be used as vaccines. In particular, the invention provides VZV and HSV antigens. In some embodiments, the antigens are specific to VZV and HSV-1 as compared to HSV-2. The pharmaceutical compositions can be used both prophylactically and therapeutically. The invention additionally provides methods, including methods for preventing and treating VZV and HSV infection, for killing VZV and HSV-infected cells, for inhibiting viral replication, for enhancing secretion of antiviral and/or immunomodulatory lymphokines, and for enhancing production of VZV- and HSV-specific antibody. For preventing and treating VZV and HSV infection, for enhancing secretion of antiviral and/or immunomodulatory lymphokines, for enhancing production of VZV- and HSV-specific antibody, and generally for stimulating and/or augmenting VZV- and HSV-specific immunity, the method comprises administering to a subject a polypeptide, polynucleotide, recombinant virus, AFC, immune cell or composition of the invention. The methods for killing VZV-infected and HSV-infected cells and for inhibiting viral replication comprise contacting a VZV-infected and/or HSV-infected cell with an immune cell of the invention. The immune cell of the invention is one that has been stimulated by an antigen of the invention or by an APC that presents an antigen of the invention. One format for presenting an antigen of the invention makes use of replication-competent or replication-incompetent, or appropriately killed, whole virus, such as VZV or HSV, that has been engineered to present one or more antigens of the invention. A method for producing immune cells of the invention is also provided. The method comprises contacting an immune cell with an APC, preferably a dendritic cell that has been modified to present an antigen of the invention. In a preferred embodiment, the immune cell is a T cell such as a CD4+ or CD8+ T cell.

In one embodiment, the VZV or HSV polypeptide comprises multiple epitopes, as set forth in Table 1, wherein the epitopes may be from the same VZV or HSV protein or from more than one VZV or HSV protein. The VZV or HSV polypeptide comprising one or more epitopes of the invention can comprise a fragment of a full-length VZV or HSV protein, or the full-length VZV or HSV protein. In some embodiments, multiple VZV or HSV polypeptides are provided together within a single composition, within a kit, or within a larger polypeptide. In one embodiment, the invention provides a multi-epitopic or multi-valent vaccine.

Specific VZV and HSV antigens and epitopes that have been identified by the method of the invention include those listed in Table 1. In one embodiment, the VZV or HSV polypeptide comprises multiple epitopes, as set forth in Table 1, wherein the epitopes may be from the same VZV or HSV protein or from more than one VZV or HSV protein. The VZV or HSV polypeptide comprising one or more epitopes of the invention can comprise a fragment of a full-length VZV or HSV protein, or the full-length VZV or HSV protein. In some embodiments, multiple VZV or HSV polypeptides are provided together within a single composition, within a kit, or within a larger polypeptide. In one embodiment, the invention provides a multi-epitopic or multi-valent vaccine.

In another embodiment, the VZV or HSV polypeptide comprises one or more type-specific VZV or HSV-1 (versus HSV-2) epitopes as identified in Table 1. In an alternative embodiment, the VZV or HSV polypeptide comprises one or more type-common (HSV-1 and HSV-2) epitopes. In a further embodiment, the VZV HSV polypeptide comprises a combination of type-common and type-specific epitopes.

In some embodiments, the selection of a combination of epitopes and/or antigens to be included within a single composition and/or polypeptide is guided by optimization of population coverage with respect to HLA alleles. For example, each epitope restricted by HLA allele A*0201 will cover 40-50% of most ethnic groups. By adding epitopes restricted by A*0101 (20%), A*2402 (-5-25%), B*0702 (10-15%), and A*29 (5-10%), one can, in the aggregate, cover more people. In one embodiment, the HSV polypeptide comprises one or more of the epitopes as associated with HLA allele A*0201. In a further embodiment, the HSV polypeptide comprises epitopes associated with one or more of the HLA alleles, A*0101, A*0201, A*2402, A*2902, and B*0702. As is understood by those skilled in the art, these HLA alleles, or HLA alleles that are biologically expected to bind to peptide epitopes restricted by these HLA alleles, cover 80-90% of the human population in most major ethnic and racial groups.

In one embodiment, the VZV polypeptide comprises one or more of ORF55, ORF42/ORF45, ORF40, ORF38, ORF36, ORF31, ORF29, ORF24, and ORF19, not necessarily in that order, in another embodiment, the VZV polypeptide comprises all of the epitopes listed in Table 1, not necessarily in the order listed. In one embodiment, HSV polypeptide comprises one or more of UL5, UL15, UL19, UL21, UL23, UL27, UL29, UL34, UL39, UL40, US8, and RS1, not necessarily in that order. In another embodiment, the HSV polypeptide comprises all of the epitopes listed in Table 1, not necessarily in the order listed. In one embodiment, the polypeptide comprises one or more of VZV ORF55, VZV ORF42/ORF45, VZV ORF40, VZV ORF38, VZV ORF38, VZV ORF31, VZV ORF29, VZV ORF24, VZV ORF19, HSV UL5, HSV UL15, HSV UL19, HSV UL21, HSV UL23, HSV UL27, HSV UL29, HSV UL34, HSV UL39, HSV UL40, HSV US8, and HSV RS1, not necessarily in that order. In another embodiment, the polypeptide comprises all of VZV ORF55, VZV ORF42/ORF45, VZV ORF40, VZV ORF38, VZV ORF36, VZV ORF31, VZV ORF29, VZV ORF24, VZV ORF19, HSV UL5, HSV UL15, HSV UL19, HSV UL21, HSV UL23, HSV UL27, HSV UL29, HSV UL34, HSV UL39, HSV UL40, HSV US8, and HSV RS1 as listed in Table 1, not necessarily in the order listed. The selection of particular combinations of antigens and/or epitopes can be guided by the data described in the figures and tables. For example, antigens that exhibit desirable characteristics and/or those that include multiple immunogenic epitopes can be combined in a single composition and/or polypeptide.

Diseases to be prevented or treated using compositions and methods of the invention include diseases associated with varicella zoster virus infection and/or herpes virus infection. In one embodiment, the diseases are associated with VZV and/or HSV-1 infection. VZV infections have considerable medical impact. For example, chickenpox and shingles can lead to death. HSV-1 infections have considerable medical impact. Examples include neonatal HSV-1 encephalitis and visceral infection leading to death or brain damage, HSV-1 encephalitis in adults, and a wide spectrum of HSV eye infections including acute retinal necrosis (ARN) and herpetic stromal keratitis (HSK). Some compositions of the invention are suitable for treating or preventing conditions resulting from infection with VZV and/or HSV-1 and conditions resulting from infection with HSV-2, Compositions can be administered to patients who may be or may become infected with either or all of VZV, HSV-1 and HSV-2.

The invention provides compositions comprising the VZV and HSV antigens and epitopes identified herein. Also provided is an isolated polynucleotide that encodes a polypeptide of the invention, and a composition comprising the polynucleotide. The invention provides a recombinant virus genetically modified to express a polynucleotide of the invention, and a composition comprising the recombinant virus. In one embodiment, the recombinant virus is vaccinia virus, canary pox virus, VZV, HSV, lentivirus, retrovirus or adenovirus. A composition of the invention can be a pharmaceutical composition, optionally comprising a pharmaceutically acceptable carrier and/or an adjuvant.

### BRIEF DESCRIPTION OF THE FIGURES

FIG. 1, Dose response for CD8 T cell responses for HSV-1 peptides of UL48 (identical in HSV-2). The 9mer at amino acids 160-168 (158-166 in HSV-2) is very active.

FIG. 2. Reactivity of CD8 T cells at 1 μg/ml for the VZV homolog of HSV UL48 peptide tested in FIG. 1.

FIG. 3. Alignment of amino acid sequences of three human alpha herpes viruses for HSV UL48 and VZV homolog. SEQ ID NOs: 55-57, respectively. Box indicates location of cross-reactive epitope.

FIG. 4. VZV-HSV cross-reactive CD8 T-cell epitopes for A*2902-restricted responses. Responders enriched from PBMC by DC cross-presentation of HSV-1/CD137 selection. APC are autologous CFSE-dump-gated PBMC. Peptides tested @1 μg/ml. Numbers are % cells in quadrants. ORF names use individual virus schemes. Note that mock-stimulated cells are 2.4% responsive=background. In the top row, both the HSV-1 and VZV peptide homolog are stimulatory. In the second row, both the HSV and VZV homologs are stimulatory. SEB=positive control.

FIG. 5. VZV-HSV cross-reactive CD8 T-cell epitopes for A*0201-restricted responses. Responders enriched from PBMC by DC cross-presentation of HSV-110D137 selection. APC are autologous CFSE-dump-gated PBMC. Peptides tested @1 μg/ml. Numbers are % cells in quadrants. ORF names use individual virus schemes. Background is lower (compared to FIG. 4) for mock. Note in top row, VZV and HSV-1 homologs both positive. In bottom row, note that VZV HSV1 HSV2 and also EBV are positive. SEB=positive control.

FIG. 6. CD4 T cell responses to VZV peptides (top bars of each panel) and their homologs in HSV 1 and HSV 2 (lower). Note that in lower panel, the T cells react to both 388-402 and 396-410 but cross reactivity is only to the 388-402 region (using VZV numbers).

FIGS. 7A-7B. Titration of CD4+ T-cell activating VZV peptides and HSV1/2 homologues.

FIG. 8, Bar graph illustrating that CD8 T-cells, which recognized both the HSV-1 and VZV epitopes, were able to recognize the full length viral gene,

FIG. 9, VZV protein subunits recognized by T cells before and after an adult shingles prevention dose of the FDA approved vOKA.

### DETAILED DESCRIPTION OF THE INVENTION

The invention provides HSV and VZV antigens that are useful for the prevention and treatment of HSV and/or VZV infection, and more particularly, specific epitopes that elicit immune responses that are cross-reactive between HSV-1, HSV-2 and VZV. Disclosed herein are antigens and/or their constituent epitopes. In some embodiments, T-cells having specificity for antigens of the invention have demonstrated cytotoxic activity against virally infected cells and/or whole virus. The identification of immunogenic antigens responsible for T-cell specificity facilitates the development of improved anti-viral therapeutic and prophylactic strategies. Compositions containing epitopes or polynucleotides encoding epitopes of the invention provide effectively targeted vaccines for prevention and treatment of alphaherpesvirus infection.

### Definitions

All scientific and technical terms used in this application have meanings commonly used in the art unless otherwise specified. As used in this application, the following words or phrases have the meanings specified.

As used herein, “polypeptide” includes proteins, fragments of proteins, and peptides, whether isolated from natural sources, produced by recombinant techniques or chemically synthesized. Polypeptides of the invention typically comprise at least about 6 amino acids, and can be at least about 15 amino acids. Typically, optimal immunological potency for peptide epitopes is obtained with lengths of 8-10 amino acids. Those skilled in the art also recognize that additional adjacent sequence from the original (native) protein can be included, and is often desired, in an immunologically effective polypeptide suitable for use as a vaccine. This adjacent sequence can be from 1, 2, 3, 4, 5, 6, 7, 8, 9 or 10 amino acids in length to as much as 15, 20, 25, 30, 35, 40, 45, 50, 75 or 100 amino acids in length or more.

As used herein, particularly in the context of polypeptides of the invention, “consisting essentially of” means the polypeptide consists of the recited amino acid sequence and, optionally, adjacent amino acid sequence. The adjacent sequence typically consists of additional, adjacent amino acid sequence found in the full length antigen, but variations from the native antigen can be tolerated in this adjacent sequence while still providing an immunologically active polypeptide.

As used herein, “multi-epitope polypeptide” means a polypeptide comprising two or more non-identical epitopes. The epitopes can be from the same or different proteins, and/or from the same or different organism. Optionally, the polypeptide may comprise more than one copy of a particular epitope, and/or more than one variant of a particular epitope. The multi-epitope polypeptide is 12 to 1200 amino acids in length. In some embodiments, the multi-epitope polypeptide is up to 600 amino acids in length.

In some embodiments, the multi-epitope polypeptide is not conjugated to and is devoid of a carrier fusion protein. In other embodiments, the multi-epitope polypeptide further comprises a carrier sequence, whereby the peptide epitopes are inserted within a sequence of a carrier polypeptide or are coupled to a carrier sequence. In some embodiments, the multi-epitope polypeptide is produced as a recombinant fusion protein comprising a carrier sequence,

As used herein, a “spacer” refers to a bond, an amino acid, or a peptide comprising at least two amino acids. A spacer is typically not more than 25 amino acids in length. In some embodiments, the spacer comprises 1 to 4 neutral amino acids. In some embodiments, the spacer comprises adjacent native sequence of the epitope's sequence of origin, where, for example, the native sequence facilitates presentation of epitope for correct processing.

As used herein, “epitope” refers to a molecular region of an antigen capable of eliciting an immune response and of being specifically recognized by the specific immune T-cell produced by such a response. Another term for “epitope” is “determinant” or “antigenic determinant”. Those skilled in the art often use the terms epitope and antigen interchangeably in the context of referring to the determinant against which an immune response is directed.

As used herein, “HSV polypeptide” includes HSV-1 and HSV-2, unless otherwise indicated. References to amino acids of HSV-1 proteins or polypeptides are based on the genomic sequence information regarding HSV-1 (strain 17+) as described in McGeoch et al., 1988, J. Gen. Virol. 69:1531-1574; GenBank Accession No. JN555585,1. References to amino acids of HSV-2 proteins or polypeptides are based on the genomic sequence information regarding HSV-2 as described in A. Dolan et al., 1998, J. Virol. 72(3):2010-2021; GenBank Accession No. JN561323.2.

As used herein, “VZV” refers to varicella zoster virus, also known as Human herpes virus 3 (HHV-3). References to amino acids of VZV proteins or polypeptides are based on the genomic sequence information regarding VZV as described in Davison & Scott, 1986, J. Gen, Virol. 67(9):1759-1816; GenBank Accession No, NC_001348.1.

As used herein, “substitutional variant” refers to a molecule having one or more amino acid substitutions or deletions in the indicated amino acid sequence, yet retaining the ability to be “immunologically active”, or specifically recognized by an immune cell. The amino acid sequence of a substitutional variant is preferably at least 80% identical to the native amino acid sequence, or more preferably, at least 90% identical to the native amino acid sequence. Typically, the substitution is a conservative substitution.

One method for determining whether a molecule is “immunologically active”, “immunologically effective”, or can be specifically recognized by an immune cell, is the cytotoxicity assay described in D. M. Koelle et al., 1997, Human Immunol. 53:195-205. Other methods for determining whether a molecule can be specifically recognized by an immune cell are described in the examples provided hereinbelow, including the ability to stimulate secretion of interferon-gamma or the ability to lyse cells presenting the molecule. An immune cell will specifically recognize a molecule when, for example, stimulation with the molecule results in secretion of greater interferon-gamma than stimulation with control molecules. For example, the molecule may stimulate greater than 5 pg/ml, or preferably greater than 10 pg/ml, interferon-gamma secretion, whereas a control molecule will stimulate less than 5 pg/ml interferon-gamma. Proliferation assays for confirming CD4 T-cell epitopes are described in Laing, et al., 2015, J. Infect. Dis. Doi: 10.1093/infdis/jiv165.

As used herein, “vector” means a construct, which is capable of delivering, and preferably expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

As used herein, “expression control sequence” means a nucleic acid sequence that directs transcription of a nucleic acid. An expression control sequence can be a promoter, such as a constitutive or an inducible promoter, or an enhancer. The expression control sequence is operably linked to the nucleic acid sequence to be transcribed.

The term “nucleic acid” or “polynucleotide” refers to a deoxyribonucleotide or ribonucleotide polymer in either single- or double-stranded form, and unless otherwise limited, encompasses known analogs of natural nucleotides that hybridize to nucleic acids in a manner similar to naturally occurring nucleotides.

As used herein, “antigen-presenting cell” or “ARC” means a cell capable of handling and presenting antigen to a lymphocyte. Examples of APCs include, but are not limited to, macrophages, Langerhans-dendritic cells, follicular dendritic cells, B cells, monocytes, fibroblasts and fibrocytes. Dendritic cells (also referred to as “DCs”) are a preferred type of antigen presenting cell. Dendritic cells are found in many non-lymphoid tissues but can migrate via the afferent lymph or the blood stream to the 1-dependent areas of lymphoid organs. In non-lymphoid organs, dendritic cells include Langerhans cells and interstitial dendritic cells. In the lymph and blood, they include afferent lymph veiled cells and blood dendritic cells, respectively. In lymphoid organs, they include lymphoid dendritic cells and interdigitating cells.

As used herein, “modified” to present an epitope refers to antigen-presenting cells (APCs) that have been manipulated to present an epitope by natural or recombinant methods. For example, the APCs can be modified by exposure to the isolated antigen, alone or as part of a mixture, peptide loading, or by genetically modifying the ARC to express a polypeptide that includes one or more epitopes.

As used herein, “pharmaceutically acceptable salt” refers to a salt that retains the desired biological activity of the parent compound and does not impart any undesired toxicological effects. Examples of such salts include, but are not limited to, (a) acid addition salts formed with inorganic acids, for example hydrochloric acid, hydrobromic acid, sulfuric acid, phosphoric acid, nitric acid and the like; and salts formed with organic acids such as, for example, acetic acid, oxalic acid, tartaric acid, succinic acid, maleic acid, furmaric acid, gluconic acid, citric acid, malic acid, ascorbic acid, benzoic acid, tannic acid, pamoic acid, alginic acid, polyglutamic acid, naphthalenesulfonic acids, naphthalenedisulfonic acids, polygalacturonic acid; (b) salts with polyvalent metal cations such as zinc, calcium, bismuth, barium, magnesium, aluminum, copper, cobalt, cadmium, and the like; or (c) salts formed with an organic cation formed from N,N′-dibenzylethylenediamine or ethylenediamine; or (d) combinations of (a) and (b) or (c), e.g., a zinc tannate salt; and the like. The preferred acid addition salts are the trifluoroacetate salt and the acetate salt.

As used herein, “pharmaceutically acceptable carrier” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline or normal (0.9%) saline.

Compositions comprising such carriers are formulated by well known conventional methods (see, for example, Remington's Pharmaceutical Sciences, 18th edition, A. Gennaro, ed., Mack Publishing Co., Easton, Pa., 1990).

As used herein, “adjuvant” includes adjuvants commonly used in the art to facilitate the stimulation of an immune response. Examples of adjuvants include, but are not limited to, helper peptide; aluminum salts such as aluminum hydroxide gel (alum) or aluminum phosphate; Freund's Incomplete Adjuvant and Complete Adjuvant (Difco Laboratories, Detroit, Mi); Merck Adjuvant 65 (Merck and Company, Inc., Rahway, N.J.); AS-2 (Smith-Kline Beecham); QS-21 (Aquilla); MPL or 3d-MPL (Corixa Corporation, Hamilton, Mont.); LEIF; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A; muramyl tripeptide phosphatidyl ethanolamine or an immunostimulating complex, including cytokines (e.g., GM-CSF or interleukin-2, -7 or -12) and immunostimulatory DNA sequences. In some embodiments, an adjuvant such as a helper peptide or cytokine can be provided via a polynucleotide encoding the adjuvant. Representative examples of such adjuvants for use in polynucleotide vaccines include, but are not limited to, ubiquitin and toll-like receptor (TLR) ligands.

As used herein, “a” or “an” means at least one, unless clearly indicated otherwise.

As used herein, to “prevent” or “protect against” a condition or disease means to hinder, reduce or delay the onset or progression of the condition or disease.

### Overview

Specific VZV and HSV-1 homologous antigens and epitopes are listed in Table 1, Table 2 provides the corresponding HSV-1 and VZV gene and protein names. Each gene is a row. Most rows have an entry for both HSV-1 and VZV. Some rows do not have a VZV gene homolog or an HSV gene homolog. Thus, cross reactivity is not possible for these genes as they do not exist in one or the other virus,

### HSV & VZV Genes and Proteins

In one embodiment, the invention provides an isolated herpes simplex virus (HSV) or varicella zoster virus (VZV) polypeptide. The polypeptide comprises at least one HSV or VZV protein or a fragment thereof. In one embodiment, the fragment is selected from those listed in the Tables and/or figures herein. In one embodiment, the fragment is a peptide selected from those listed in Table 1.

In one embodiment, the invention provides an isolated polynucleotide encoding an alphaherpesvirus multi-epitope polypeptide. The alphaherpesvirus multi-epitope polypeptide comprises a plurality of alphaherpesvirus peptide epitopes linked in a series, wherein each epitope in the series is linked to an adjacent epitope by a spacer. The spacer comprises a bond, an amino acid, or a peptide comprising at least two amino acids. The spacer can be selected to facilitate epitope processing and/or cleavage between two or more epitopes. A spacer is typically not more than 25 amino acids in length. In some embodiments, the spacer comprises 1 to 4 neutral amino acids. In some embodiments, the spacer comprises adjacent native sequence of the epitopes sequence of origin, where, for example, the native sequence facilitates presentation of epitope for correct processing. Optimization of poly-epitope immunogens is described, for example, in Reguzova et al., 2015, PLoS One 10(3):e0116412 (PMC4364888). In some embodiments, the spacer comprises a cleavable sequence. In one embodiment, a cleavable spacer is cleaved by intracellular enzymes. In a more specific embodiment, the cleavable spacer comprises a protease specific cleavable sequence.

The plurality of alphaherpesvirus peptide epitopes comprises at least one epitope described herein, and typically comprises at least one epitope selected from Table 1. In one embodiment, the plurality of alphaherpesvirus peptide epitopes comprises epitopes that elicit T-cell reactivity to HSV-1, HSV-2, and VZV. In another embodiment, the plurality of alphaherpesvirus peptide epitopes comprises epitopes that elicit T-cell reactivity to HSV-1, HSV-2, or VZV. In one embodiment, the plurality of peptide epitopes comprises at least two epitopes selected from Table 1. In another embodiment, the plurality of peptides epitopes comprises 3, 4, 5, 6, 7, 8, 9, 10, 11, 12, 15, or 18 epitopes selected from Table 1. In one embodiment, the plurality of peptide epitopes comprises no more than 20 epitopes described herein. In another embodiment, the plurality of peptide epitopes comprises no more than 15 epitopes described herein. In another embodiment, the plurality of peptide epitopes comprises no more than 10 epitopes described herein.

In one embodiment, the plurality of peptide epitopes comprises the epitope ELRAREEXY, wherein X is A or S (SEQ ID NO: 58). In another embodiment, the plurality of peptide epitopes comprises the epitope QPMRLYSTCLYHPNA (SEQ ID NO: 36), or another epitope of Table 1 that exhibits a high degree of similarity across VZV, HSV-1 and/or HSV-2. In another embodiment, the plurality of peptide epitopes comprises at least one epitope identified in Table 1 as a CD4 epitope and at least one epitope identified in Table 1 as a CD8 epitope. In another embodiment, the plurality of peptide epitopes comprises one or more epitopes identified in Table 3 as cross-reactive between VZV and HSV-1. In another embodiment, the plurality of peptide epitopes comprises one or more epitopes identified in Table 3 as having at least 4 amino acids in common between VZV and HSV-1.

In another embodiment, the plurality of peptide epitopes comprises at least one epitope identified herein as an HLA A*0201 epitope, at least one epitope identified herein as an HLA A*2902 epitope, and, optionally, at least one epitope identified herein as an HLA B*1502 epitope, in another embodiment, the plurality of peptide epitopes comprises at least one epitope identified in Table 1 as an HSV-1 epitope, at least one epitope identified in Table 1 as an HSV-2 epitope, and at least one epitope identified in Table 1 as a VZV epitope. Other combinations of epitopes are contemplated, including any combination of 2, 3, 4, 5, 6, 7, 8 or more epitopes listed in Table 1, optionally together with one or more epitopes not described herein.

In one embodiment, the invention provides a recombinant alphaherpesvirus multi epitope polypeptide, such as a polypeptide encoded by a polynucleotide described herein. Also provided is a multi-epitope p*peptide produced by a recombinant virus genetically modified to express an alphaherpesvirus multi-epitope polypeptide described herein.

TABLE 1
Cross-Reactive Epitopes of HSV and VZV
CD4 epitopes

| | | | | | HSV-1 | | | HSV-2 peptide |
|---|---|---|---|---|---|---|---|---|
| | | | | | peptide (SEQ | | | (SEQ ID NOs: |
| | | | | Location | ID NOs: 13-24) | | Location | 25-36) Bold |
| | | VZV peptide | | (aa) | HSV-1 | | (aa) | same as HSV1, |
| | Location (aa) | (SEQ ID NOs: | | in HSV-1 | difference | | in HSV-2 | underlined |
| VZV ORF | in VZV protein | 1-12) | HSV-1 ORF | protein | bold | HSV-2 ORF | protein | unique to HSV2 |
| ORF | 233-247 | STGDIIYMS | UL | 290-304 | ATGDFVYMSP | UL | 285-299 | ATGDFVYMSP |
| 31 | | PFFGLR | 27 | | FYGYR | 27 | | FYGYR |
| ORF | 237-251 | IIYMSPFFG | UL | 294-308 | FVYMSPFYGY | UL | 289-303 | FVYMSPFYGY |
| 31 | | LRDGAY | 27 | | REGSH | 27 | | REGSH |
| ORF | 527-541 | TRQPIGVFG | UL | 529-529 | ARGAIGVFGT | UL | 529-543 | ARGAIGVFGTM |
| 29 | | TMNSQY | 29 | | MNSMY | 29 | | NSAY |
| ORF | 531-545 | IGVFGTMNS | UL | 533-547 | IGVFGTMNSM | UL | 533-547 | IGVFGTMNSAY |
| 29 | | QYSDCD | 29 | | YSDCD | 29 | | SDCD |
| ORF | 594-608 | YGLYNSQFL | UL | 956-970 | HGLRNSQFVA | UL | 961-975 | HGLRNSQFIAL |
| 19 | | ALMPTV | 39 | | LMPTA | 39 | | MPTA |
| ORF | 598-612 | NSQFLALMP | UL | 960-974 | NSQFVALMPT | UL | 965-979 | NSQFIALMPTA |
| 19 | | TVSSAQ | 39 | | AASAQ | 39 | | ASAQ |
| ORF | 57-69 | FIFTFLSAA | UL | 85-97 | FLFAFLSAAD | UL | 82-94 | FLFAFLSAADD |
| 18 | | DDLV | 40 | | DLV | 40 | | LV |
| ORF | 85-97 | IHHYYIEQE | UL | 113-123 | ILHYYVEQEC | UL | 110-122 | ILHYYVEQECI |
| 18 | | CIEV | 40 | | IEV | 40 | | EV |
| ORF | 165-177 | SSFAAIAYL | UL | 193-205 | ASFAAIAYLR | UL | 190-202 | ASFAAIAYLRT |
| 18 | | RNNG | 40 | | TNN | 40 | | NN |
| ORF 9 | 177-189 | NKRVFCEAV | UL | 197-209 | NKRVFCAAVG | UL | 197-209 | NKRVFCAAVGR |
| | | RRVA | 49 | | RLA | 49 | | LA |
| ORF | 84-95 | PYIKIQNTG | UL | 83-94 | PYLRIQNTGV | UL | 83-94 | PYLRVQNTGVS |
| 24 | | VSV | 34 | | SV | 34 | | V |
| ORF | 388-402 | QPMRLYSTC | US 8 | 272-286 | AEMRIYESCL | US 8 | 267-281 | ADMRIYEACLY |
| 68 | | LYHPNA | | | YHPQL | | | HPQL |

CD8 epitopes

| | VZV peptide (SEQ ID | | | | | | HSV-2 peptide (SEQ ID | |
|---|---|---|---|---|---|---|---|---|
| | NOs: 37-42) | | | HSV-1 peptide | | | NOs. 49-54) | |
| | Difference from HSV-1 | | | (SEQ ID NOs: | | | Differences from HSV-1 | |
| | in bold | | | 43-48) | | | is underlined | |
| ORF | 361-369 | FLMEDQTLL | UL | 367-375 | FLWEDQTLL | UL | 372-380 | FLWEDQTLL |
| 34¹ | | | 25 | | | 25 | | |
| ORF | 156-164 | ILIEGIFFV | UL | 184-192 | ILIEGIFFA | UL | 181-189 | ILIEGVFFA |
| 18¹ | | | 40 | | | 40 | | |
| ORF | 232-240 | AVLCLYLMY | UL | 235-243 | AVLCLYLLY | UL | 240-248 | AVLCLYLLY |
| 34² | | | 25 | | | 25 | | |
| ORF | 893-901 | YMANLILKY | UL | 895-903 | YMANQILRY | UL | 895-903 | YMANQILRY |
| 29² | | | 29 | | | 29 | | |
| ORF | 163-175 | VELRAREEA | UL | 159-171 | AELRAREESY | UL | 157-169 | GELRAREESYR |
| 10³ | | YTKL | 48 | | RTV | 48 | | TV |
| ORF | 164-172 | ELRAREEAY | UL | 160-168 | ELRAREESY | UL | 158-166 | ELRAREESY |
| 10³ | | | 48 | | | 48 | | |

¹HLA A*0201
²HLA A*2902
³HLA B*1502

TABLE 2
HSV & VZV Genes & Proteins

| HSV gene | VZV gene | Protein (NCBI) |
|---|---|---|
| UL1 | ORF60 | gL |
| UL2 | ORF59 | uracil-DNA glycosylase |
| UL3 | ORF58 | nuclear protein UL3 |
| UL4 | ORF56 | nuclear protein UL4 |
| UL5 | ORF55 | helicase-primase helicase subunit |
| UL6 | ORF54 | capsid portal protein |
| UL7 | ORF53 | tegument protein UL7 |
| UL8 | ORF52 | helicase-primase subunit |
| UL9 | ORF51 | DNA replication origin-binding helicase |
| UL10 | ORF50 | DNA replication origin-binding helicase |
| UL11 | ORF49 | myristylated tegument protein |
| UL12 | ORF48 | deoxyribonuclease |
| UL13 | ORF47 | tegument serine/threonine protein kinase |
| UL14 | ORF46 | tegument protein UL14 |
| UL15 | ORF42/ORF45 | DNA packaging terminase subunit 1 |
| UL16 | ORF44 | tegument protein UL16 |
| UL17 | ORF43 | DNA packaging tegument protein UL17 |
| UL18 | ORF41 | capsid triplex subunit (VP23) |
| UL19 | ORF40 | major capsid protein (VP5) |
| UL20 | ORF39 | envelope protein UL20 |
| UL21 | ORF38 | tegument protein UL21 |
| UL22 | ORF37 | gH |
| UL23 | ORF36 | thymidine kinase |
| UL24 | ORF35 | nuclear protein UL24 |
| UL25 | ORF34 | DNA packaging tegument protein UL25 |
| UL26 | ORF33 | capsid maturation protease |
| UL26.5 | ORF33.5 | major capsid scaffold protein |
| UL27 | ORF31 | gB |
| UL28 | ORF30 | DNA packaging terminase subunit 2 |
| UL29 | ORF29 | single-stranded DNA-binding protein |
| UL30 | ORF28 | DNA polymerase catalytic subunit |
| UL31 | ORF27 | nuclear egress lamina protein |
| UL32 | ORF26 | DNA packaging protein UL32 |
| UL33 | ORF25 | DNA packaging protein UL33 |
| UL34 | ORF24 | nuclear egress membrane protein |
| UL35 | ORF23 | small capsid protein (VP26) |
| UL36 | ORF22 | large tegument protein (VP1-2) |
| UL37 | ORF21 | tegument protein UL37 |
| UL38 | ORF20 | capsid triplex subunit 1 (VP19C) |
| UL39 | ORF19 | ribonucleotide reductase subunit 1 |
| UL40 | ORF18 | ribonucleotide reductase subunit 2 |
| UL41 | ORF17 | tegument virion host shutoff protein |
| | | (vhs) |
| UL42 | ORF16 | DNA polymerase processivity subunit |
| UL43 | ORF15 | envelope protein UL43 |
| UL44 | ORF14 | gC |
| UL45 | no VZV gene | membrane protein UL45 |
| UL46 | ORF12 | tegument protein (VP11/12) |
| UL47 | ORF11 | tegument protein (VP13/14) |
| UL48 | ORF10 | transactivating tegument protein (VP16) |
| UL49 | ORF9 | tegument protein (VP22) |
| UL49.5 | ORF9α | gN |
| UL50 | ORF8 | deoxyuridine triphosphatase |
| UL51 | ORF7 | tegument protein UL51 |
| UL52 | ORF6 | helicase-primase primase subunit |
| UL53 | ORF5 | gK |
| UL54 | ORF4 | multifunctional expression regulator |
| | | (ICP27) |
| UL55 | ORF3 | nuclear protein UL55 |
| UL56 | ORF0 | membrane protein UL56 |
| US1 | ORF63 | regulatory protein (ICP22) |
| US2 | no VZV gene | virion protein US2 |
| US3 | ORF66 | serine/threonine protein kinase US3 |
| US4 | no VZV gene | gG |
| US5 | no VZV gene | gJ |
| US6 | no VZV gene | gD |
| US7 | ORF67 | gI |
| US8 | ORF68 | gE |
| US8.5 | no VZV gene | membrane protein US8A |
| US9 | ORF65 | membrane protein US9 |
| US10 | ORF64 | virion protein US10 |
| US11 | no VZV gene | tegument protein US11 |
| US12 | no VZV gene | TAP transporter inhibitor (ICP47) |
| RL1 | no VZV gene | ICP34.5 |
| RL2 | ORF61 | ICP0 |
| RS1 | ORF62 | ICP4 |
| no HSV gene | ORF1 | membrane protein V1 |
| no HSV gene | ORF2 | myristylated tegument protein CIRC |
| no HSV gene | ORF13 | thymidylate synthase |
| no HSV gene | ORF32 | phosphoprotein 32 (function unknown) |
| no HSV gene | ORF57 | tegument protein |

A fragment of the invention consists of less than the complete amino acid sequence of the corresponding protein, but includes the recited epitope or antigenic region. As is understood in the art and confirmed by assays conducted using fragments of widely varying lengths, additional sequence beyond the recited epitope can be included without hindering the immunological response. A fragment of the invention can be as few as 8 amino acids in length, or can encompass 10%, 20%, 30%, 40%, 50%, 60%, 70%, 80%, 90% or 95% of the full length of the protein.

The optimal length for the polypeptide of the invention will vary with the context and objective of the particular use, as is understood by those in the art. In some vaccine contexts, a full-length protein or large portion of the protein (e.g., up to 100 amino acids, 150 amino acids, 200 amino acids, 250 amino acids or more provides optimal immunological stimulation, while in others, a short polypeptide (e.g., less than 50 amino acids, 40 amino acids, 30 amino acids, 20 amino acids, 15 amino acids or fewer) comprising the minimal epitope and/or a small region of adjacent sequence facilitates delivery and/or eases formation of a fusion protein or other means of combining the polypeptide with another molecule or adjuvant.

A polypeptide for use in a composition of the invention comprises an HSV polypeptide that contains an epitope or minimal stretch of amino acids sufficient to elicit an immune response. These polypeptides typically consist of such an epitope and, optionally, adjacent sequence. Those skilled in the art are aware that the HSV epitope can still be immunologically effective with a small portion of adjacent HSV or other amino acid sequence present. Accordingly, a typical polypeptide of the invention will consist essentially of the recited epitope and have a total length of up to 15, 20, 25 or 30 amino acids.

A typical embodiment of the invention is directed to a polypeptide consisting essentially of an epitope listed in Table 1, More specifically, a polypeptide consisting of an epitope listed in Table 1 and, optionally, up to 15 amino acids of adjacent native sequence. In some embodiments, the polypeptide is fused with or co-administered with a heterologous peptide. The heterologous peptide can be another epitope or unrelated sequence. The unrelated sequence may be inert or it may facilitate the immune response. In some embodiments, the epitope is part of a multi-epitopic vaccine, in which numerous epitopes are combined in one polypeptide.

In general, polypeptides (including fusion proteins) and polynucleotides as described herein are isolated. An “isolated” polypeptide or polynucleotide is one that is removed from its original environment. The isolated molecule can then be of particular use because multiple copies are available in a substantially purified preparation, enabling utilization of the molecule in ways not possible without isolation and/or recombinant production. For example, a naturally occurring protein is isolated if it is separated from some or all of the coexisting materials in the natural system. An isolated RSV polypeptide of the invention is one that has been isolated, produced or synthesized such that it is separate from a complete, native herpes simplex virus, although the isolated polypeptide may subsequently be introduced into a recombinant virus. A recombinant virus that comprises an isolated polypeptide or polynucleotide of the invention is an example of subject matter provided by the invention. Preferably, such isolated polypeptides are at least about 90% pure, more preferably at least about 95% pure and most preferably at least about 99% pure. A polynucleotide is considered to be isolated if, for example, it is cloned into a vector that is not part of the natural environment.

The polypeptide can be isolated from its naturally occurring form, produced by recombinant means or synthesized chemically. Recombinant polypeptides encoded by DNA sequences described herein can be readily prepared from the DNA sequences using any of a variety of expression vectors known to those of ordinary skill in the art. Expression may be achieved in any appropriate host cell that has been transformed or transfected with an expression vector containing a DNA molecule that encodes a recombinant polypeptide. Suitable host cells include prokaryotes, yeast and higher eukaryotic cells. Preferably the host cells employed are E. coli, yeast or a mammalian cell line such as Cos or CHO. Supernatants from the soluble host/vector systems that secrete recombinant protein or polypeptide into culture media may be first concentrated using a commercially available filter. Following concentration, the concentrate may be applied to a suitable purification matrix such as an affinity matrix or an ion exchange resin. Finally, one or more reverse phase HPLC steps can be employed to further puffy a recombinant polypeptide.

Fragments and other variants having less than about 100 amino acids, and generally less than about 50 amino acids, may also be generated by synthetic means, using techniques well known to those of ordinary skill in the art. For example, such polypeptides may be synthesized using any of the commercially available solid-phase techniques, such as the Merrifield solid-phase synthesis method, wherein amino acids are sequentially added to a growing amino acid chain (Merrifield, 1963, J. Am. Chem. Soc. 85:2146-2149). Equipment for automated synthesis of polypeptides is commercially available from suppliers such as Perkin Elmer/Applied BioSystems Division (Foster City, Calif.), and may be operated according to the manufacturer's instructions.

Variants of the polypeptide for use in accordance with the invention can have one or more amino acid substitutions, deletions, additions and/or insertions in the amino acid sequence indicated that result in a polypeptide that retains the ability to elicit an immune response to alphaherpesvirus-infected cells. Such variants may generally be identified by modifying one of the polypeptide sequences described herein and evaluating the reactivity of the modified polypeptide using a known assay such as a T cell assay described herein. Polypeptide variants preferably exhibit at least about 70%, more preferably at least about 90%, and most preferably at least about 95% identity to the identified polypeptides. These amino acid substitutions include, but are not necessarily limited to, amino acid substitutions known in the art as “conservative”. Those skilled in the art recognize that any substitutions are preferably made in amino acids outside of the minimal epitope identified herein.

A “conservative” substitution is one in which an amino acid is substituted for another amino acid that has similar properties, such that one skilled in the art of peptide chemistry would expect the secondary structure and hydropathic nature of the polypeptide to be substantially unchanged. Amino acid substitutions may generally be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gin, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes. In a preferred embodiment, variant polypeptides differ from a native sequence by substitution, deletion or addition of five amino acids or fewer. Variants may also (or alternatively) be modified by, for example, the deletion or addition of amino acids that have minimal influence on the immunogenicity, secondary structure and hydropathic nature of the polypeptide.

One can readily confirm the suitability of a particular variant by assaying the ability of the variant polypeptide to elicit an immune response. The ability of the variant to elicit an immune response can be compared to the response elicited by the parent polypeptide assayed under identical circumstances. One example of an immune response is a cellular immune response. The assaying can comprise performing an assay that measures T cell stimulation or activation. Examples of T cells include CD4 and CD8 T cells.

One example of a T cell stimulation assay is a cytotoxicity assay, such as that described in Koelle, DM et al., Human Immunol. 1997, 53; 195-205. In one example, the cytotoxicity assay comprises contacting a cell that presents the antigenic viral peptide in the context of the appropriate HLA molecule with a T cell, and detecting the ability of the T cell to kill the antigen presenting cell. Cell killing can be detected by measuring the release of radioactive ⁵¹Cr from the antigen presenting cell. Release of ⁵¹Cr into the medium from the antigen presenting cell is indicative of cell killing. An exemplary criterion for increased killing is a statistically significant increase in counts per minute (cpm) based on counting of ⁵¹Cr radiation in media collected from antigen presenting cells admixed with T cells as compared to control media collected from antigen presenting cells admixed with media.

### Fusion Proteins

The polypeptide can be a fusion protein. In one embodiment, the fusion protein is soluble. A soluble fusion protein of the invention can be suitable for injection into a subject and for eliciting an immune response. Within certain embodiments, a polypeptide can be a fusion protein that comprises multiple polypeptides as described herein, or that comprises at least one polypeptide as described herein and an unrelated sequence. In one example, the fusion protein comprises a HSV epitope described herein (with or without flanking adjacent native sequence) fused with non-native sequence. A fusion partner may, for example, assist in providing T helper epitopes (an immunological fusion partner), preferably T helper epitopes recognized by humans, or may assist in expressing the protein (an expression enhancer) at higher yields than the native recombinant protein. Certain preferred fusion partners are both immunological and expression enhancing fusion partners. Other fusion partners may be selected so as to increase the solubility of the protein or to enable the protein to be targeted to desired intracellular compartments. Still further fusion partners include affinity tags, which facilitate purification of the protein.

Fusion proteins may generally be prepared using standard techniques, including chemical conjugation. Preferably, a fusion protein is expressed as a recombinant protein, allowing the production of increased levels, relative to a non-fused protein, in an expression system. Briefly, DNA sequences encoding the polypeptide components may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion protein that retains the biological activity of both component polypeptides.

A peptide linker sequence may be employed to separate the first and the second polypeptide components by a distance sufficient to ensure that each polypeptide folds into its secondary and tertiary structures. Such a peptide linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., 1985, Gene 40:39-46; Murphy et al., 1986, Proc. Natl. Acad. Sci. USA 83:8258-8262; U.S. Pat. No. 4,935,233 and U.S. Pat. No. 4,751,180, The linker sequence may generally be from 1 to about 50 amino acids in length. Linker sequences are not required when the first and second polypeptides have non-essential N-terminal amino acid regions that can be used to separate the functional domains and prevent steric interference.

The ligated DNA sequences are operably linked to suitable transcriptional or translational regulatory elements. The regulatory elements responsible for expression of DNA are located 5′ to the DNA sequence encoding the first polypeptides. Similarly, stop codons required to end translation and transcription termination signals are present 3′ to the DNA sequence encoding the second polypeptide.

Fusion proteins are also provided that comprise a polypeptide of the present invention together with an unrelated immunogenic protein. Preferably the immunogenic protein is capable of eliciting a recall response. Examples of such proteins include tetanus, tuberculosis and hepatitis proteins (see, for example, Stoute et al., 1997, New Engl. J. Med., 336:86-9).

Within preferred embodiments, an immunological fusion partner is derived from protein D, a surface protein of the gram-negative bacterium Haemophilus influenza B (WO 91/18926). Preferably, a protein D derivative comprises approximately the first third of the protein (e.g., the first N-terminal 100-110 amino acids), and a protein D derivative may be lipidated. Within certain preferred embodiments, the first 109 residues of a Lipoprotein D fusion partner is included on the N-terminus to provide the polypeptide with additional exogenous T-cell epitopes and to increase the expression level in E. coli (thus functioning as an expression enhancer). The lipid tail ensures optimal presentation of the antigen to antigen presenting cells. Other fusion partners include the non-structural protein from influenza virus, NS1 (hemaglutinin). Typically, the N-terminal 81 amino acids are used, although different fragments that include 1-helper epitopes may be used.

In another embodiment, the immunological fusion partner is the protein known as LYTA, or a portion thereof (preferably a C-terminal portion). LYTA is derived from Streptococcus pneumoniae, which synthesizes an N-acetyl-L-alanine amidase known as amidase LYTA (encoded by the LytA gene; Gene 43:265-292, 1986). LYTA is an autolysin that specifically degrades certain bonds in the peptidoglycan backbone. The C-terminal domain of the LYTA protein is responsible for the affinity to the choline or to some choline analogues such as DEAE. This property has been exploited for the development of E. coli C-LYTA expressing plasmids useful for expression of fusion proteins. Purification of hybrid proteins containing the C-LYTA fragment at the amino terminus has been described (see Biotechnology 10:795-798, 1992). Within a preferred embodiment, a repeat portion of LYTA may be incorporated into a fusion protein. A repeat portion is found in the C-terminal region starting at residue 178. A particularly preferred repeat portion incorporates residues 188-305.

In some embodiments, it may be desirable to couple a therapeutic agent and a polypeptide of the invention, or to couple more than one polypeptide of the invention. For example, more than one agent or polypeptide may be coupled directly to a first polypeptide of the invention, or linkers that provide multiple sites for attachment can be used. Alternatively, a carrier can be used. Some molecules are particularly suitable for intercellular trafficking and protein delivery, including, but not limited to, VP22 (Elliott and O′Hare, 1997, Cell 88:223-233; see also Kim et al., 1997, J. Immunol. 159:1666-1668; Rojas et al., 1998, Nature Biotechnology 16:370; Kato et al., 1998, FEBS Lett. 427(2):203-208; Nagahara et al., 1998, Nature Med. 4(12):1449-1452).

A carrier may bear the agents or polypeptides in a variety of ways, including covalent bonding either directly or via a linker group. Suitable carriers include proteins such as albumins (e.g., U.S. Pat. No. 4,507,234, to Kato et al,), peptides and polysaccharides such as aminodextran (e.g., U.S. Pat. No. 4,699,784, to Shih et al.). A carrier may also bear an agent by noncovalent bonding or by encapsulation, such as within a liposome vesicle (e.g., U.S. Pat. Nos. 4,429,008 and 4,873,088).

Polynucleotides. Vectors, Host Cells and Recombinant Viruses

The invention provides polynucleotides that encode one or more polypeptides of the invention. The polynucleotide can be included in a vector. The vector can further comprise an expression control sequence operably linked to the polynucleotide of the invention. In some embodiments, the vector includes one or more polynucleotides encoding other molecules of interest. In one embodiment, the polynucleotide of the invention and an additional polynucleotide can be linked so as to encode a fusion protein.

Within certain embodiments, polynucleotides may be formulated so to permit entry into a cell of a mammal, and expression therein. Such formulations are particularly useful for therapeutic purposes, as described below. Those of ordinary skill in the art will appreciate that there are many ways to achieve expression of a polynucleotide in a target cell, and any suitable method may be employed. For example, a polynucleotide may be incorporated into a viral vector such as, but not limited to, adenovirus, adeno-associated virus, retrovirus, vaccinia or a pox virus (e.g., avian pox virus). Techniques for incorporating DNA into such vectors are well known to those of ordinary skill in the art. A retroviral vector may additionally transfer or incorporate a gene for a selectable marker (to aid in the identification or selection of transduced cells) and/or a targeting moiety, such as a gene that encodes a ligand for a receptor on a specific target cell, to render the vector target specific. Targeting may also be accomplished using an antibody, by methods known to those of ordinary skill in the art.

The invention also provides a host cell transformed with a vector of the invention. The transformed host cell can be used in a method of producing a polypeptide of the invention. The method comprises culturing the host cell and recovering the polypeptide so produced. The recovered polypeptide can be purified from culture supernatant.

Vectors of the invention can be used to genetically modify a cell, either in vivo, ex vivo or in vitro Several ways of genetically modifying cells are known, including transduction or infection with a viral vector either directly or via a retroviral producer cell, calcium phosphate precipitation, fusion of the recipient cells with bacterial protoplasts containing the DNA, treatment of the recipient cells with liposomes or microspheres containing the DNA, DEAE dextran, receptor-mediated endocytosis, electroporation, micro-injection, and many other techniques known to those of skill. See, e.g., Sambrook et al. Molecular Cloning—A Laboratory Manual (2nd ed.) 1-3, 1989; and Current Protocols in Molecular Biology, F. M. Ausubel et al., eds., Greene Publishing Associates, Inc, and John Wiley & Sons, Inc., (1994 Supplement).

Examples of viral vectors include, but are not limited to retroviral vectors based on, e.g., HIV, SIV, and murine retroviruses, gibbon ape leukemia virus and other viruses such as adeno-associated viruses (AAVs) and adenoviruses. (Miller et al. 1990, Mol. Cell Biol. 10:4239; J. Koberg 1992, NIH Res. 4:43, and Cornetta et al. 1991, Hum. Gene Ther. 2:215). Widely used retroviral vectors include those based upon murine leukemia virus (MuLV), gibbon ape leukemia virus (GaLV), ecotropic retroviruses, simian immunodeficiency virus (SIV), human immunodeficiency virus (HIV), and combinations. See, e.g. Buchscher et al. 1992, J. Virol. 66(5):2731-2739; Johann et al, 1992, J. Virol. 66(5):1635-1640; Sommerfelt et al. 1990, Virol. 176:58-59; Wilson et al. 1989, J. Virol. 63:2374-2378; Miller et al. 1991, J. Virol. 65:2220-2224, and Rosenberg and Feud 1993 in Fundamental Immunology, Third Edition, WE. Paul (ed.) Raven Press, Ltd., New York and the references therein; Miller et al. 1990, Mol. Cell. Biol. 10:4239; R. Kolberg 1992, J. NIH Res. 4:43; and Cornetta et al. 1991, Hum. Gene Ther, 2:215.

In vitro amplification techniques suitable for amplifying sequences to be subcloned into an expression vector are known. Examples of such in vitro amplification methods, including the polymerase chain reaction (PCR), ligase chain reaction (LCR), Qβ-replicase amplification and other RNA polymerase mediated techniques (e.g., NASBA), are found in Sambrook et al. 1989, Molecular Cloning—A Laboratory Manual (2nd Ed) 1-3; and U.S. Pat. No. 4,683,202; PCR Protocols A Guide to Methods and Applications (Innis et al. eds.) Academic Press Inc, San Diego, Calif. 1990. Improved methods of cloning in vitro amplified nucleic acids are described in US. Pat. No. 5,426,039.

The invention additionally provides a recombinant microorganism genetically modified to express a polynucleotide of the invention. The recombinant microorganism can be useful as a vaccine, and can be prepared using techniques known in the art for the preparation of live attenuated vaccines. Examples of microorganisms for use as live vaccines include, but are not limited to, viruses and bacteria. In a preferred embodiment, the recombinant microorganism is a virus. Examples of suitable viruses include, but are not limited to, vaccinia virus and other poxviruses.

### Compositions

The invention provides compositions that are useful for treating and preventing HSV and/or VZV infection. The compositions can be used to inhibit viral replication and to kill virally-infected cells. In one embodiment, the composition is a pharmaceutical composition. The composition can comprise a therapeutically or prophylactically effective amount of a polypeptide, polynucleotide, recombinant virus, APC or immune cell of the invention. An effective amount is an amount sufficient to elicit or augment an immune response, e.g., by activating T cells. One measure of the activation of T cells is a cytotoxicity assay, as described in D. M. Koelle et al., 1997, Human Immunol. 53:195-205. In some embodiments, the composition is a vaccine.

The composition can optionally include a carrier, such as a pharmaceutically acceptable carrier. Pharmaceutically acceptable carriers are determined in part by the particular composition being administered, as well as by the particular method used to administer the composition. Accordingly, there is a wide variety of suitable formulations of pharmaceutical compositions of the present invention. Formulations suitable for parenteral administration, such as, for example, by intraarticular (in the joints), intravenous, intramuscular, intradermal, intraperitoneal, and subcutaneous routes, and carriers include aqueous isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, preservatives, liposomes, microspheres and emulsions.

The composition of the invention can further comprise one or more adjuvants. Examples of adjuvants include, but are not limited to, helper peptide, alum, Freund's, muramyl tripeptide phosphatidyl ethanolamine or an immunostimulating complex, including cytokines. In some embodiments, such as with the use of a polynucleotide vaccine, an adjuvant such as a helper peptide or cytokine can be provided via a polynucleotide encoding the adjuvant. Vaccine preparation is generally described in, for example, M. F. Powell and M. J. Newman, eds., “Vaccine Design (the subunit and adjuvant approach),” Plenum Press (NY, 1995). Pharmaceutical compositions and vaccines within the scope of the present invention may also contain other compounds, which may be biologically active or inactive. For example, one or more immunogenic portions of other viral antigens may be present, either incorporated into a fusion polypeptide or as a separate compound, within the composition or vaccine. Additional information about peptide vaccines can be found in Li et al., 2014. Vaccines 2: 515-536, and about adjuvant use with a Herpes zoster vaccine in Lai et al., 2015, New Engl J Med DOI: 10.1056/NEJMoa1501184.

A pharmaceutical composition or vaccine may contain DNA encoding one or more of the polypeptides of the invention, such that the polypeptide is generated in situ. As noted above, the DNA may be present within any of a variety of delivery systems known to those of ordinary skill in the art, including nucleic acid expression systems, bacteria and viral expression systems. Numerous gene delivery techniques are well known in the art, such as those described by Rolland, 1998, Grit. Rev. Therap. Drug Carrier Systems 15:143-198, and references cited therein. Appropriate nucleic acid expression systems contain the necessary DNA sequences for expression in the patient (such as a suitable promoter and terminating signal). Bacterial delivery systems involve the administration of a bacterium (such as Bacillus-Calmette-Guerrin) that expresses an immunogenic portion of the polypeptide on its cell surface or secretes such an epitope. In a preferred embodiment, the DNA may be introduced using a viral expression system (e.g., vaccinia or other pox virus, retrovirus, or adenovirus), which may involve the use of a non-pathogenic (defective), replication competent virus. Suitable systems are disclosed, for example, in Fisher-Hoch et al., 1989, Proc. Natl. Acad. Sci. USA 86:317-321; Flexner et al., 1989, Ann. My Acad. Sci. 569:86-103; Flexner et al., 1990, Vaccine 8:17-21; U.S. Pat. Nos. 4,603,112, 4,769,330, and 5,017,487; WO 89/01973; U.S. Pat. No. 4,777,127; GB 2,200,651; EP 0,345,242; WO 91102805; Berkner, 1988, Biotechniques 6:616-627; Rosenfeld et al., 1991, Science 252:431-434; Kolls et al., 1994, Proc. Natl. Acad. Sci. USA 91:215-219; Kass-Eisler et al., 1993, Proc. Natl. Acad. Sci. USA 90:11498-11502; Guzman et al., 1993, Circulation 88:2838-2848; and Guzman et al., 1993, Cir. Res. 73:1202-1207. Techniques for incorporating DNA into such expression systems are well known to those of ordinary skill in the art. The DNA may also be “naked,” as described, for example, in Ulmer et al., 1993, Science 259:1745-1749 and reviewed by Cohen, 1993, Science 259:1691-1692. The uptake of naked DNA may be increased by coating the DNA onto biodegradable beads, which are efficiently transported into the cells.

While any suitable carrier known to those of ordinary skill in the art may be employed in the pharmaceutical compositions of this invention, the type of carrier will vary depending on the mode of administration. Compositions of the present invention may be formulated for any appropriate manner of administration, including for example, topical, oral, nasal, intravenous, intracranial, intraperitoneal, subcutaneous or intramuscular administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactate polyglycolate) may also be employed as carriers for the pharmaceutical compositions of this invention. Suitable biodegradable microspheres are disclosed, for example, in US. Pat. Nos. 4,897,268 and 5,075,109.

Such compositions may also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, chelating agents such as EDTA or glutathione, adjuvants (e.g., aluminum hydroxide) and/or preservatives. Alternatively, compositions of the present invention may be formulated as a lyophilizate. Compounds may also be encapsulated within liposomes via known methods.

A variety of adjuvants may be employed in the vaccines of this invention. Most adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Suitable adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); aluminum salts such as aluminum hydroxide gel (alum) or aluminum phosphate; salts of calcium, iron or zinc; an insoluble suspension of acylated tyrosine acylated sugars; cationically or anionically derivatized polysaccharides; polyphosphazenes biodegradable microspheres; monophosphoryl lipid A and gild A. Cytokines, such as GM CSF or interleukin-2, -7, or -12, may also be used as adjuvants.

Within the vaccines provided herein, the adjuvant composition is preferably designed to induce an immune response predominantly of the Th1 type. High levels of Th1-type cytokines (e.g., IFN-γ, IL-2 and IL-12) tend to favor the induction of cell mediated immune responses to an administered antigen. In contrast, high levels of Th2-type cytokines (e.g., IL-4, 1L-5, 1L-6, 1L-10 and TNF-β) tend to favor the induction of humoral immune responses. Following application of a vaccine as provided herein, a patient will support an immune response that includes Th1- and Th2-type responses. Within a preferred embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see Mosmann and Coffman, 1989, Ann. Rev. Immunol. 7:145-173.

Preferred adjuvants for use in eliciting a predominantly Th1-type response include, for example, a combination of monophosphoryl lipid A, preferably 3-de-O-acylated monophosphoryl lipid A (3D-MPL), together with an aluminum salt. MPL™ adjuvants are available from Corixa Corporation (see US Pat. Nos. 4,436,727; 4,877,611; 4,866,034 and 4,912,094). CpG-containing oligonucleotides (in which the CpG dinucleotide is unmethylated) also induce a predominantly Th1 response. Such oligonucleotides are well known and are described, for example, in WO 96/02555, Another preferred adjuvant is a saponin, preferably QS21, which may be used alone or in combination with other adjuvants. For example, an enhanced system involves the combination of a monophosphoryl lipid A and saponin derivative, such as the combination of QS21 and 3D-MPL as described in WO 94/00153, or a less reactogenic composition where the QS21 is quenched with cholesterol, as described in WO 96/33739. Other preferred formulations comprise an oil-in-water emulsion and tocopherol. A particularly potent adjuvant formulation involving QS21, 3D-MPL and tocopherol in an oil-in-water emulsion is described in WO 95/17210. Another adjuvant that may be used is AS-2 (Smith-Kline Beecham). Any vaccine provided herein may be prepared using well known methods that result in a combination of antigen, immune response enhancer and a suitable carrier or excipient.

The compositions described herein may be administered as part of a sustained release formulation (i.e., a formulation such as a capsule or sponge that effects a slow release of compound following administration). Such formulations may generally be prepared using well known technology and administered by, for example, oral, rectal or subcutaneous implantation, or by implantation at the desired target site. Sustained-release formulations may contain a polypeptide, polynucleotide or antibody dispersed in a carrier matrix and/or contained within a reservoir surrounded by a rate controlling membrane. Carriers for use within such formulations are biocompatible, and may also be biodegradable; preferably the formulation provides a relatively constant level of active component release. The amount of active compound contained within a sustained release formulation depends upon the site of implantation, the rate and expected duration of release and the nature of the condition to be treated or prevented.

A variety of delivery vehicles may be employed within pharmaceutical compositions and vaccines to facilitate production of an antigen-specific immune response that targets HSV-infected cells. Delivery vehicles include antigen presenting cells (APCs), such as dendritic cells, macrophages, B cells, monocytes and other cells engineered to be efficient APCs. Such cells may, but need not, be genetically modified to increase the capacity for presenting antigen, to improve activation and/or maintenance of the T cell response, to have antiviral effects per se and/or to be immunologically compatible with the receiver (i.e., matched HLA haplotype), APCs may generally be isolated from any of a variety of biological fluids and organs, including tumor and peritumoral tissues, and may be autologous, allogeneic, syngeneic or xenogeneic cells.

Certain preferred embodiments of the present invention use dendritic cells or progenitors thereof as antigen-presenting cells, Dendritic cells are highly potent APCs (Banchereau and Steinman, Nature 392:245-251, 1998) and have been shown to be effective as a physiological adjuvant for eliciting prophylactic or therapeutic immunity (see Timmerman and Levy, Ann. Rev. Med. 50:507-529, 1999). In general, dendritic cells may be identified based on their typical shape (stellate in situ, with marked cytoplasmic processes (dendrites) visible in vitro) and based on the lack of differentiation markers of B cells (CD19 and CD20), T cells (CD3), monocytes (CD14) and natural killer cells (CD56), as determined using standard assays. Dendritic cells may be engineered to express specific cell-surface receptors or ligands that are not commonly found on dendritic cells in vivo or ex vivo, and such modified dendritic cells are contemplated by the invention. As an alternative to dendritic cells, secreted vesicles antigen-loaded dendritic cells (called exosomes) may be used within a vaccine (Zitvogel et al., 1998, Nature Med. 4:594-600),

Dendritic cells and progenitors may be obtained from peripheral blood, bone marrow, tumor-infiltrating cells, peritumoral tissues-infiltrating cells, lymph nodes, spleen, skin, umbilical cord blood or any other suitable tissue or fluid. For example, dendritic cells may be differentiated ex vivo by adding a combination of cytokines such as GM-CSF, IL-4, IL-13 and/or TNFα to cultures of monocytes harvested from peripheral blood. Alternatively, CD34 positive cells harvested from peripheral blood, umbilical cord blood or bone marrow may be differentiated into dendritic cells by adding to the culture medium combinations of GM-CSF, IL-3, TNFα, CD40 ligand, LPS, flt3 ligand and/or other compound(s) that induce maturation and proliferation of dendritic cells.

APCs may be transfected with a polynucleotide encoding a polypeptide (or portion or other variant thereof) such that the polypeptide, or an immunogenic portion thereof, is expressed on the cell surface. Such transfection may take place ex vivo, and a composition or vaccine comprising such transfected cells may be used for therapeutic purposes, as described herein. Alternatively, a gene delivery vehicle that targets a dendritic or other APC may be administered to a patient, resulting in transfection that occurs in vivo. In vivo and ex vivo transfection of dendritic cells, for example, may generally be performed using any methods known in the art, such as those described in WO 97/24447, or the gene gun approach described by Mahvi et al., 1997, Immunology and Cell Biology 75:456-460. Antigen loading of dendritic cells may be achieved by incubating dendritic cells or progenitor cells with the tumor polypeptide, DNA (naked or within a plasmid vector) or RNA; or with antigen-expressing recombinant bacterium or viruses (e.g., vaccinia, fowlpox, adenovirus or lentivirus vectors). Prior to loading, the polypeptide may be covalently conjugated to an immunological partner that provides T cell help (e.g., a carrier molecule). Alternatively, a dendritic cell may be pulsed with a non conjugated immunological partner, separately or in the presence of the polypeptide.

### Administration of the Compositions

Treatment includes prophylaxis and therapy. Prophylaxis or treatment can be accomplished by a single direct injection at a single time point or multiple time points. Administration can also be nearly simultaneous to multiple sites. Patients or subjects include mammals, such as human, bovine, equine, canine, feline, porcine, and ovine animals as well as other veterinary subjects. Typical patients or subjects are human.

Compositions are typically administered in vivo via parenteral (e.g. intravenous, subcutaneous, and intramuscular) or other traditional direct routes, such as buccal/sublingual, rectal, oral, nasal, topical, (such as transdermal and ophthalmic), vaginal, pulmonary, intraarterial, intraperitoneal, intraocular, or intranasal routes or directly into a specific tissue.

The compositions are administered in any suitable manner, often with pharmaceutically acceptable carriers. Suitable methods of administering cells in the context of the present invention to a patient are available, and, although more than one route can be used to administer a particular cell composition, a particular route can often provide a more immediate and more effective reaction than another route.

The dose administered to a patient, in the context of the present invention should be sufficient to effect a beneficial therapeutic response in the patient over time, or to inhibit infection or disease due to infection. Thus, the composition is administered to a patient in an amount sufficient to elicit an effective immune response to the specific antigens and/or to alleviate, reduce, cure or at least partially arrest symptoms and/or complications from the disease or infection. An amount adequate to accomplish this is defined as a “therapeutically effective dose.”

The dose will be determined by the activity of the composition produced and the condition of the patient, as well as the body weight or surface areas of the patient to be treated. The size of the dose also will be determined by the existence, nature, and extent of any adverse side effects that accompany the administration of a particular composition in a particular patient. In determining the effective amount of the composition to be administered in the treatment or prophylaxis of diseases such as HSV infection, the physician needs to evaluate the production of an immune response against the virus, progression of the disease, and any treatment-related toxicity.

For example, a vaccine or other composition containing a subunit HSV/VZV protein can include 1-10,000 micrograms of HSV/VZV protein per dose. In a preferred embodiment, 10-1000 micrograms of HSV/VZV protein is included in each dose in a more preferred embodiment 10-100 micrograms of HSV/VZV protein dose. Preferably, a dosage is selected such that a single dose will suffice or, alternatively, several doses are administered over the course of several months. For compositions containing HSV/VZV polynucleotides or peptides, similar quantities are administered per dose.

In one embodiment, between 1 and 10 doses may be administered over a 52 week period. Preferably, 6 doses are administered, at intervals of 1 month, and booster vaccinations may be given periodically thereafter. Alternate protocols may be appropriate for individual patients. A suitable dose is an amount of a compound that, when administered as described above, is capable of promoting an antiviral immune response, and is at least 10-50% above the basal (i.e., untreated) level. Such vaccines should also be capable of causing an immune response that leads to an improved clinical outcome in vaccinated patients as compared to non-vaccinated patients. In general, for pharmaceutical compositions and vaccines comprising one or more polypeptides, the amount of each polypeptide present in a dose ranges from about 0.1 pg to about 5 mg per kg of host. Preferably, the amount ranges from about 10 to about 1000 μg per dose. Suitable volumes for administration will vary with the size, age and immune status of the patient, but will typically range from about 0.1 mL to about 5 mL, with volumes less than about 1 mL being most common.

Compositions comprising immune cells are preferably prepared from immune cells obtained from the subject to whom the composition will be administered. Alternatively, the immune cells can be prepared from an HLA-compatible donor. The immune cells are obtained from the subject or donor using conventional techniques known in the art, exposed to APCs modified to present an epitope of the invention, expanded ex vivo, and administered to the subject. Protocols for ex vivo therapy are described in Rosenberg et al., 1990, New England J. Med. 9:570-578. In addition, compositions can comprise APCs modified to present an epitope of the invention.

Immune cells may generally be obtained in sufficient quantities for adoptive immunotherapy by growth in vitro, as described herein. Culture conditions for expanding single antigen-specific effector cells to several billion in number with retention of antigen recognition in vivo are well known in the art. Such in vitro culture conditions typically use intermittent stimulation with antigen, often in the presence of cytokines (such as IL-2) and non-dividing feeder cells. As noted above, immunoreactive polypeptides as provided herein may be used to enrich and rapidly expand antigen-specific T cell cultures in order to generate a sufficient number of cells for immunotherapy. In particular, antigen-presenting cells, such as dendritic, macrophage, monocyte, fibroblast and/or B cells, may be pulsed with immunoreactive polypeptides or transfected with one or more polynucleotides using standard techniques well known in the art. For example, antigen-presenting cells can be transfected with a polynucleotide having a promoter appropriate for increasing expression in a recombinant virus or other expression system. Cultured effector cells for use in therapy must be able to grow and distribute widely, and to survive long term in vivo. Studies have shown that cultured effector cells can be induced to grow in vivo and to survive long term in substantial numbers by repeated stimulation with antigen supplemented with IL-2 (see, for example, Cheever et al., 1997, Immunological Reviews 157:177).

Administration by many of the routes of administration described herein or otherwise known in the art may be accomplished simply by direct administration using a needle, catheter or related device, at a single time point or at multiple time points.

### In Vivo Testing of Identified Antigens

Conventional techniques can be used to confirm the in vivo efficacy of the identified HSV/VZV antigens. For example, one technique makes use of a mouse challenge model. Those skilled in the art, however, will appreciate that these methods are routine, and that other models can be used.

Once a compound or composition to be tested has been prepared, the mouse or other subject is immunized with a series of injections. For example up to 10 injections can be administered over the course of several months, typically with one to 4 weeks elapsing between doses. Following the last injection of the series, the subject is challenged with a dose of virus established to be a uniformly lethal dose. A control group receives placebo, while the experimental group is actively vaccinated. Alternatively, a study can be designed using sublethal doses. Optionally, a dose-response study can be included. The end points to be measured in this study include death and severe neurological impairment, as evidenced, for example, by spinal cord gait. Survivors can also be sacrificed for quantitative viral cultures of key organs including spinal cord, brain, and the site of injection. The quantity of virus present in ground up tissue samples can be measured. Compositions can also be tested in previously infected animals for reduction in recurrence to confirm efficacy as a therapeutic vaccine.

Efficacy can be determined by calculating the IC50, which indicates the micrograms of vaccine per kilogram body weight required for protection of 50% of subjects from death. The IC50 will depend on the challenge dose employed. In addition, one can calculate the LD50, indicating how many infectious units are required to kill one half of the subjects receiving a particular dose of vaccine. Determination of post mortem viral titer provides confirmation that viral replication was limited by the immune system.

A subsequent stage of testing would be a vaginal inoculation challenge. For acute protection studies, mice can be used. Because they can be studied for both acute protection and prevention of recurrence, guinea pigs provide a more physiologically relevant subject for extrapolation to humans. In this type of challenge, a non-lethal dose is administered, the guinea pig subjects develop lesions that heal and recur. Measures can include both acute disease amelioration and recurrence of lesions. The intervention with vaccine or other composition can be provided before or after the inoculation, depending on whether one wishes to study prevention versus therapy.

### Methods of Treatment and Prevention

The invention provides a method for treatment and/or prevention of an alphaherpesvirus infection, such as an HSV and/or VZV infection, in a subject. The method comprises administering to the subject a composition, polynucleotide, or polypeptide of the invention. The composition, polynucleotide or polypeptide can be used as a therapeutic or prophylactic vaccine. In one embodiment, the HSV is HSV-1. Alternatively, the HSV is HSV-2, The invention additionally provides a method for inhibiting alphaherpesvirus replication, for killing alphaherpesvirus -infected cells, for increasing secretion of lymphokines having antiviral and/or immunomodulatory activity, and for enhancing production of herpes-specific antibodies. The method comprises contacting an HSV- and/or VZV-infected cell with an immune cell directed against an antigen of the invention, for example, as described in the Examples presented herein. The contacting can be performed in vitro or in vivo. In a preferred embodiment, the immune cell is a T cell. T cells include CD4 and CD8 T cells. Alternatively, the methods for inhibiting alphaherpesvirus replication, for killing alphaherpesvirus -infected cells, for increasing secretion of lymphokines having antiviral and/or immunomodulatory activity, and for enhancing production of herpes-specific antibodies can be achieved by administering a composition, polynucleotide or polypeptide of the invention to a subject. Compositions of the invention can also be used as a tolerizing agent against immunopathologic disease.

In addition, the invention provides a method of producing immune cells directed against an alphaherpesvirus, such as HSV and/or VZV. The method comprises contacting an immune cell with an alphaherpesvirus polypeptide of the invention. The immune cell can be contacted with the polypeptide via an antigen-presenting cell, wherein the antigen-presenting cell is modified to present an antigen included in a polypeptide of the invention. Preferably, the antigen-presenting cell is a dendritic cell. The cell can be modified by, for example, peptide loading or genetic modification with a nucleic acid sequence encoding the polypeptide. In one embodiment, the immune cell is a T cell. T cells include CD4 and CD8 T cells. Also provided are immune cells produced by the method. The immune cells can be used to inhibit HSV and/or VZV replication, to kill HSV- and/or VZV-infected cells, in vitro or in vivo, to increase secretion of lymphokines having antiviral and/or immunomodulatory activity, to enhance production of herpes-specific antibodies, or in the treatment or prevention of HSV and/or VZV infection in a subject.

The invention also provides a diagnostic assay. The diagnostic assay can be used to identify the immunological responsiveness of a patient suspected of having a herpetic infection and to predict responsiveness of a subject to a particular course of therapy. The assay comprises exposing T cells of a subject to an antigen of the invention, in the context of an appropriate APC, and testing for immunoreactivity by, for example, measuring IFNγ, proliferation or cytotoxicity. Suitable assays are known in the art.

### EXAMPLES

The following examples are presented to illustrate the present invention and to assist one of ordinary skill in making and using the same. The examples are not intended in any way to otherwise limit the scope of the invention.

### Example 1

### Identification of Cross-Reactivity Against Full-Length Proteins

This Example demonstrates the identification of cross reactive proteins. T-cell mixtures were created, using blood, which T-cells react to whole VZV or whole HSV-1. Table 1 above summarizes all cross-reactive epitopes described in the following examples. Table 2 above provides the HSV-1 gene name in column 2, and column 4 indicates the corresponding VZV gene number. Column 5 of Table 2 is a summary of protein function. Each gene is a row. Note that most rows have an entry for both the HSV-1 and VZV columns. Some rows, e.g. row 67, show there is no VZV gene homolog; and for row 82, there is no HSV gene homolog, etc. Thus, cross reactivity is not possible for these genes, they don't exist in one or the other virus.

Some persons studied were HSV-1-infected, and we made a T cell mixture from their blood using HSV-1 as the key tool to create the T cell mixture. For example, human subject AG13847 had a positive reaction to HSV-1 protein UL5, but not to the VZV homolog ORF55. For this theme of cross-reactivity, focus on the subjects, such as in human subject TT13850, who had a positive T cell response to both HSV-1 protein UL5 and VZV protein ORF55. The proteins that are cross-reactive (HSV/VZV) in the most humans are UL5/ORF55, UL15/ORF42/45, UL19/ORF40, UL21/ORF38, UL23/ORF36, UL27/ORF31, UL29/ORF29, UL34/ORF24, UL39/ORF19, UL40/ORF18, US8/ORF68, ICP4(RS1)/ORF62. Some of the most population prevalent cross-reactive CD4 antigens are HSV-1 UL34/VZVORF24 (4 people), HSV-1 ORF UL29/VZV ORF29 (3 people), HSV-1 ORF40/VZV ORF18 (3 people) and HSV-1 ORF ICP4 (also called RS1)/VZV OPF62 (also called ORF71 and IE62) (6 people).

For one subject, the blood from this person was treated with VZV as a key tool to create a mixture of T cells before the testing was done. For this person, HSV-1 protein UL34 and VZV protein ORF24 were both positive.

### Example 2

### Identification of Cross-Reactivity Against Discrete Peptides

This Example demonstrates the identification of cross reactive epitopes. Table 3 lists peptide epitopes recognized by cross-reactive CD4 and CD8 T-cells. Bold type in the Table indicates tetramers working directly ex vivo in peripheral blood mononuclear cells (PBMC).

TABLE 3
Well-defined VZV T-cell epitopes.

| | | | | |
|---|---|---|---|---|
| | | | | HSV-1 |
| VZV | VZV amino | CD4 vs | | similarity, |
| ORF | acids | CD8 | HLA | amino acids |

All HLA restriction-defined epitopes from literature

| | | | | |
|---|---|---|---|---|
| 4 | 256-268 | CD4 | DRB1*07 | 4 of 13 |
| 67 | 144-155 | CD4 | DRBR*04 | 6 of 12 |
| 63 | 229-243 | CD4 | DRB1*15 | none |
| 68 | 542-556 | CD4 | DRB1*1501 | 4 of 17 |
| 68 | 193-206 | CD4 | DRB1*07 | 5 of 14 |
| 68 | 281-300 | CD4 | DRB4*01 | none |
| 62 | 445-454 | CD8 | A*0201 | 4 of 10 |
| 62 | 448-457 | CD8 | A*0201 | 2 of 10 |
| 62 | 471-480 | CD8 | A*0201 | none |
| 62 | 593-601 | CD8 | A*0201 | 2 of 9 |

New epitopes (XR = HSV-1 cross-reactive)

| | | | | |
|---|---|---|---|---|
| 34 | 84-94 | CD4 | DQB1*0302 or | 9 of 11, XR |
| | | | 0501 | |
| 68 | 388-402 | CD4 | DPB1*0201 or | 8 of 15, XR |
| | | | 0301 | |
| 68 | 396-410 | CD4 | DRB1*15 | 9 of 15 |
| 29 | 893-901 | CD8 | A*2902 | 7 of 9, XR |
| 34 | 232-240 | CD8 | A*2902 | 8 of 9, XR |
| 18 | 361-369 | CD8 | A*0201 | 8 of 9, XR |
| 34 | 156-164 | CD8 | A*0201 | 8 of 9, XR |

The ORFs labeled XR are cross-reactive between VZV and HSV-1. The lower section of the Table is directed to novel epitopes.

### Example 3

### Minimal Cross-Reactive Epitope of HSV UL48/VZV ORF10

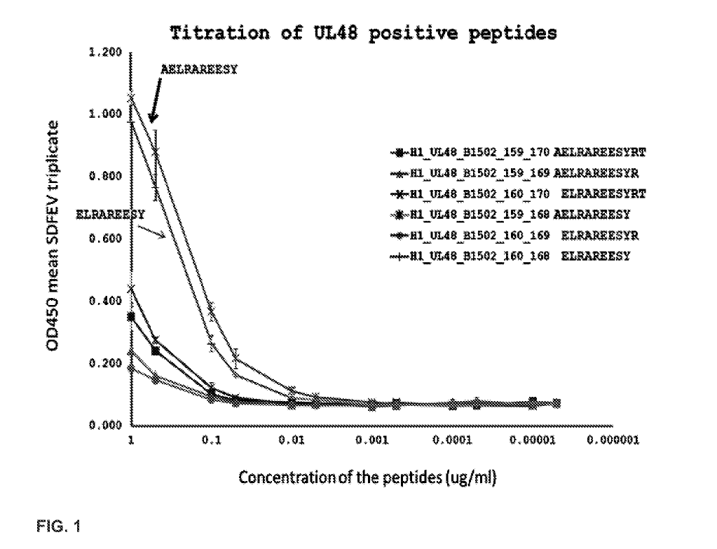

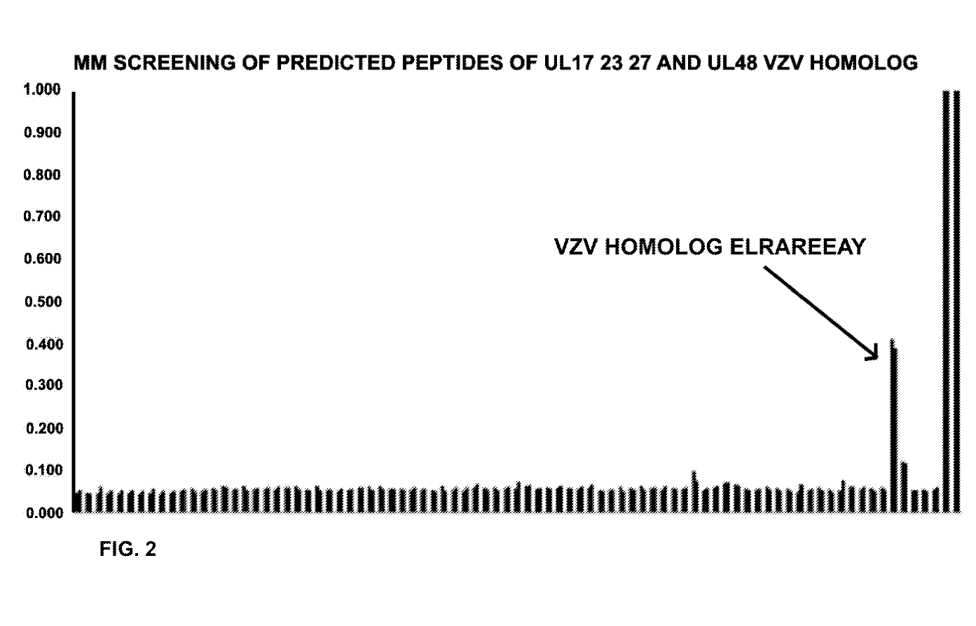

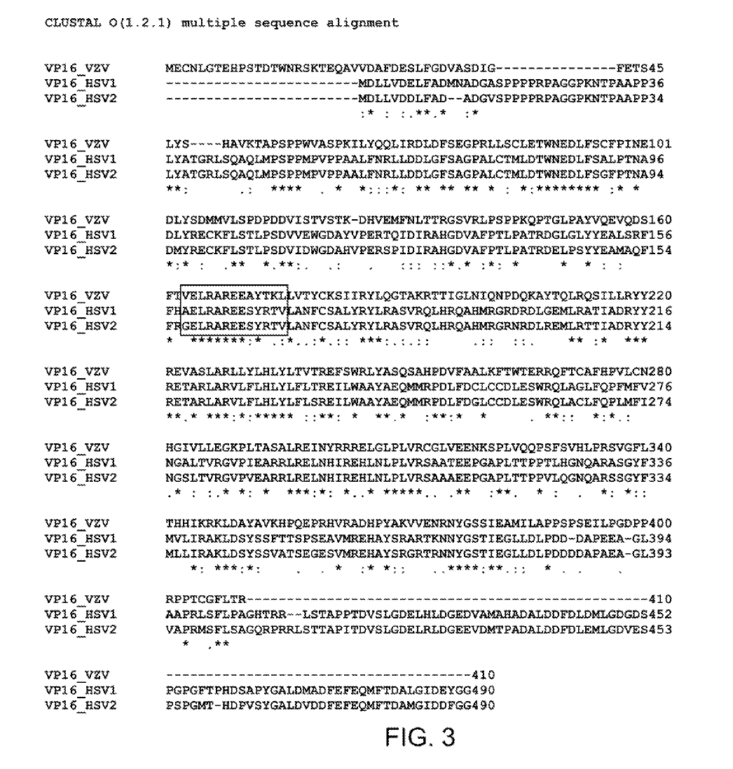

This Example demonstrates titration of UL48 positive peptides and their VZV homologs. UL48 of HSV is also known as VP16, and its VZV homolog is ORF10. FIG. 1 shows dose response curves for CD8 T cell responses for the HSV-1 peptides, which are identical in HSV-2. The 9 mer at amino acids 160-168 of HSV-1 (amino acids 158-166 of HSV-2) is very active. FIG. 2 shows reactivity at 1 μg/ml for the VZV homolog, at amino acids 164-172 of ORF10, also (+). Alignment of the amino acid sequences for the HSV-1, HSV-2, and VZV homologs are shown in FIG. 3, with the cross-reactive region boxed.

### Example 4

### HLA-Specific Responses to Cross-Reactive CD8 Epitopes

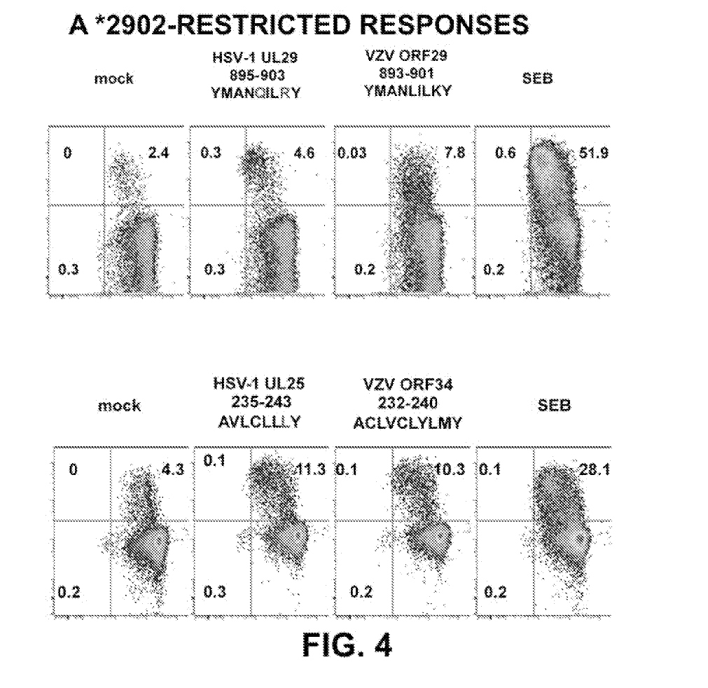

This Example demonstrates cross-reactive CD8 epitopes in the context of HLA-restriction. FIG. 4 shows VZV-HSV cross-reactive CD8 T-cell epitopes for A2902-restricted responses. Responder cells were enriched from PBMC by DC cross-presentation of HSV-1/CD137 selection. APC are autologous carboxyfluorescein succinimidyl ester (CFSE)-dump-gated PBMC. Peptides were tested at 1 μg/ml. Numbers are percent cells in quadrants. ORF names use individual virus schemes. Note that mock-stimulated cells are 2.4% responsive, as the background. In the top row, both the HSV-1 and VZV peptide homolog are stimulatory. In the second row, both the HSV and VZV homologs are stimulatory. Staphylococcal enterotoxin B (SEB) is the positive control.

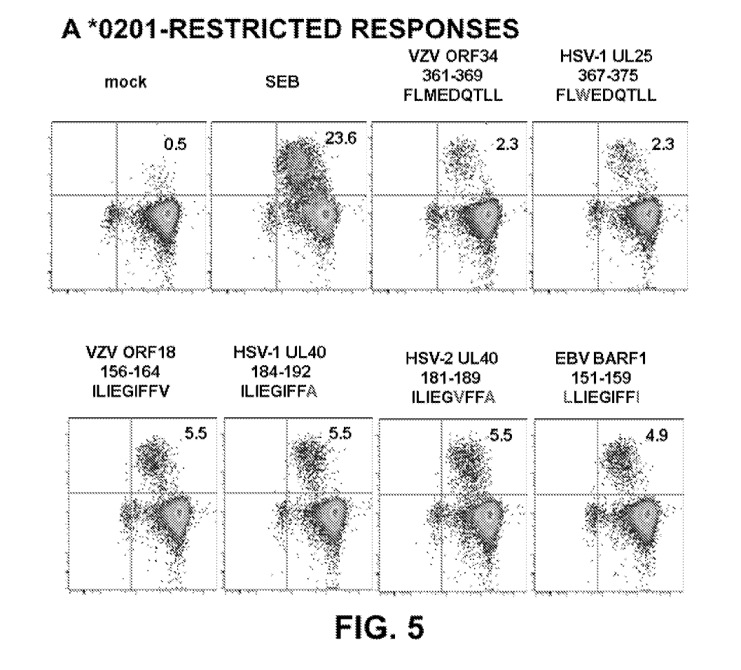

FIG. 5 shows VZV-HSV cross-reactive CD8 T-cell epitopes for A*0201-restricted responses. Responders were enriched from PBMC by DC cross-presentation of HSV-1/CD137 selection. AFC are autologous CFSE-dump-gated PBMC. Peptides tested at 1 μg/ml. Numbers are percent cells in quadrants. ORF names use individual virus schemes. The background for this set is lower (compared to FIG. 4) for mock. Note in top row, VZV and HSV-1 homologs are both positive. In bottom row, note that VZV HSV1 HSV2 and also EBV are positive. SEB is the positive control.

### Example 5

### CD4 T-Cell Responses to Cross-Reactive Epitopes

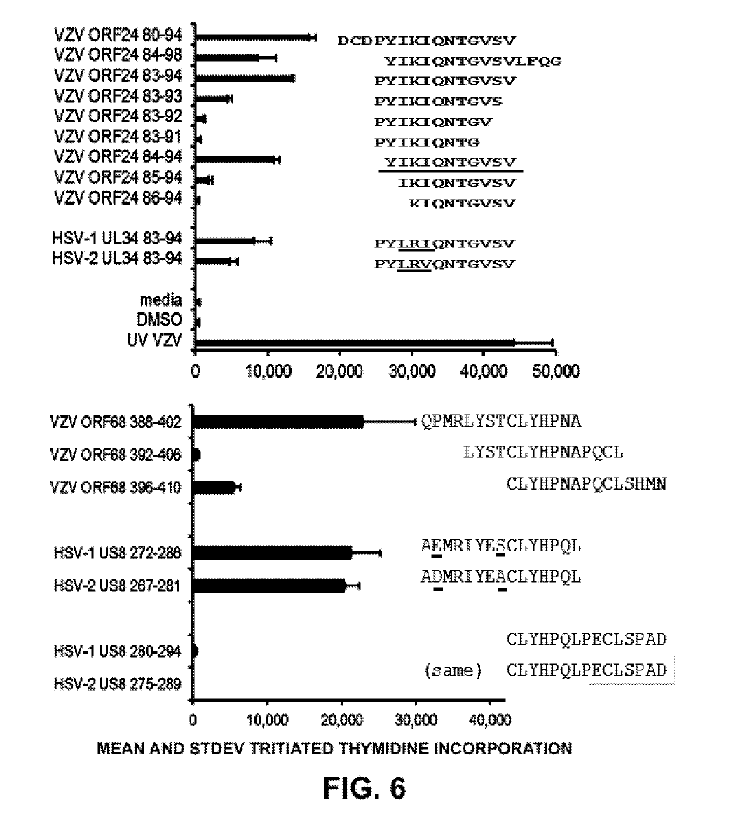

This Example demonstrates cross-reactive CD4 T cell responses to VZV peptides and their homologs in HSV 1 and HSV 2 (FIG. 6). Note that the lower panel of FIG. 6 shows that the T cells react to both 388-402 and 396-410, but cross reactivity is only to the 388-402 region (using VZV numbers). In FIG. 6, VZV and HSV-1 gene names are given along with amino acid numbers and sequences. For the upper graph in FIG. 6, the minimal active epitopes are VZV ORF24 84-94 (underlined) and HSV-1 UL34 83-94 and HSV-2 UL34 83-94. For the lower graph of FIG. 6, the sequences are as shown. Differences in amino acid sequence between the viruses are indicated with underlining. Peptide epitopes were queried with bulk CD137 high origin polyclonal T-cell line. Two discrete epitopes in VZV ORF68 (gE); one, AA 388-402, is cross-reactive with both HSV-1 and HSV-2. ORF68 396-401 is not cross-reactive with HSV. Data are mean±standard deviation, duplicate ³H thymidine incorporation; APC are LCL (lymphoblastoid cell lines).

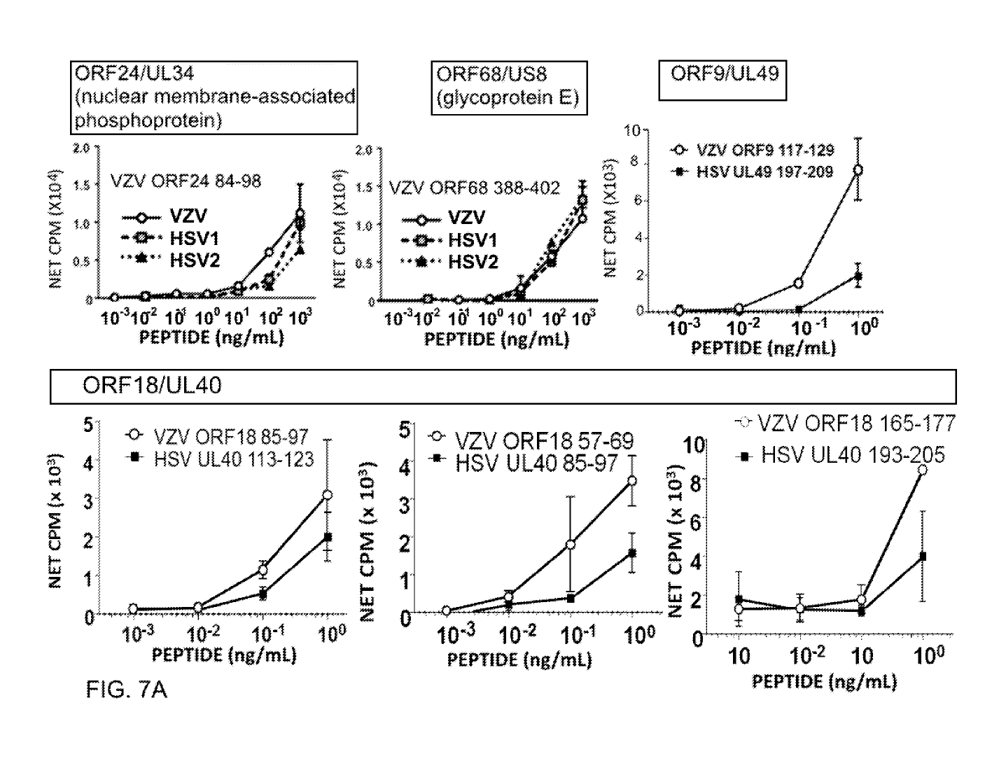

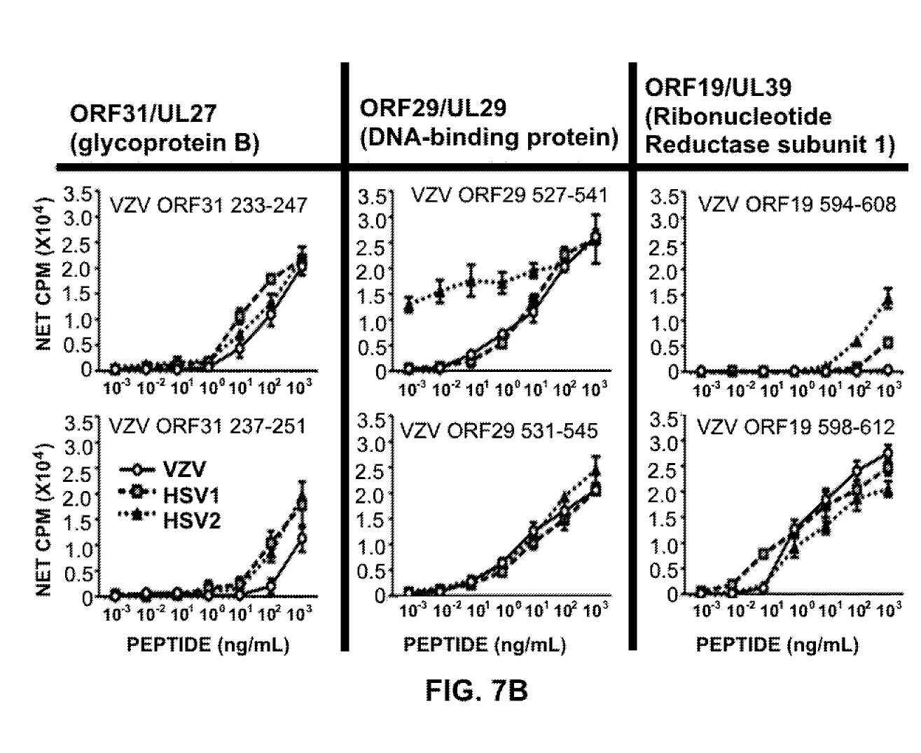

FIGS. 7A-7B are dose response curves that illustrate the titration of CD4+ T-cell activating VZV peptides and HSV1/2 homologues.

### Example 6

### CD8 T-Cell Responses to cross-Reactive Epitopes

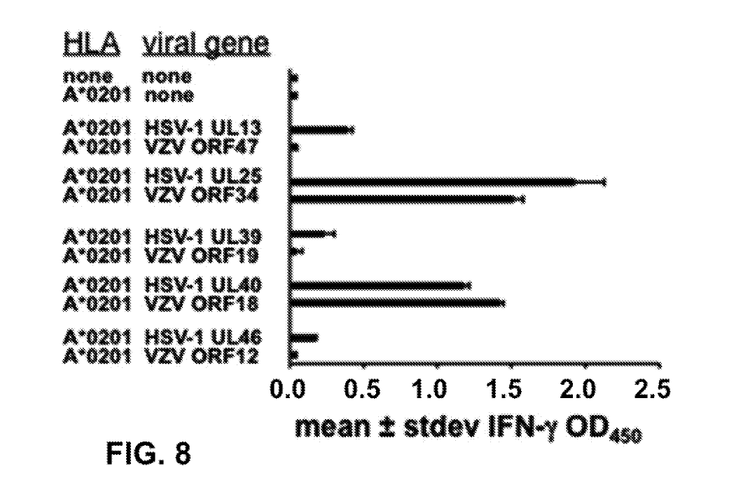

This Example demonstrates cross-reactive CD8 T cell responses. Functional capabilities of the cross-reactive CD8 T-cells are shown in FIG. 8. For the two HLA A*0201 restricted epitopes shown in FIG. 5, we tested to see if the CD8 T-cells that recognized both the HSV-1 and VZV variants of these peptides were able to recognize full length viral genes and also full actual virus. These are important improvements over just recognizing peptides. For both the VZV ORF18 (HSV UL40) and VZV ORF34 (HSV ORF25)-specific CD8 T-cells, we proved recognition of full-length viral genes.

The full length viral gene data is shown in FIG. 8. The X axis is a measure of CD8 T cell recognition. Note that artificial antigen presenting cells had to be transfected with both HLA A*0201, the population-prevalent variant of a human immune response gene, and either the HSV-1 gene or the VZV gene, in the case of double transfection, we got a strong response. In contrast for HSV-1 UL13, 39, and 46, the VZV homologs ORF 47, 19, or 12 were not cross-reactive.

Recognition of whole VZV virus was also tested. CD8 T-cells specific for HSV-1 UL25=VZV ORF34 or HSV-2 UL40=VZV ORF18 were purified from blood and contacted in cell culture with human skin cells that were either HLA A*0201 (+) or HLA A*0201 (−). The HLA 0201 genotype is required for the recognition event. The human skin cells were also either uninfected, infected with VZV vaccine strain vOKA, or infected with a wild-type (WT) circulating VZV clinical strain from a patient at the UW virology lab with shingles. The results show that for both 008 T cells specific for HSV-1 UL25/VZV ORF34, and for HSV-1 UL40/VZV ORF18, there was specific recognition and killing only of human skin cells with HLA A0201 and VZV infection. Similar killing data show that these same CD8 T-cells can selectively kill HSV-1-infected cells.

### Example 7

### T-Cell Recognition of VZV Proteins Before and After Shingles Vaccination

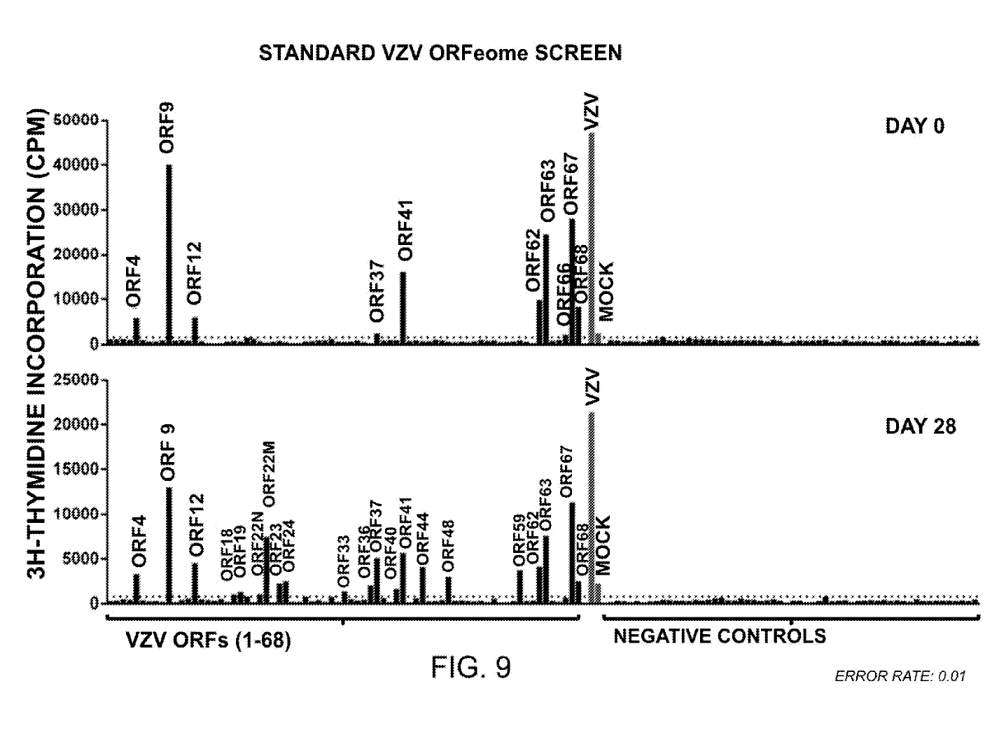

This Example demonstrates recognition of VZV protein subunits by T cells before and after an adult shingles prevention dose of the FDA approved vOKA. Nine people were studied for CD4 T cell responses to every VZV protein before and after an adult shingles prevention dose of the FDA approved vOKA. ORFs well-represented included those for regulatory proteins ORF4(ICP27) 4 subjects showed responses before & 5 after vaccination, ORF62(ICP4)—3 before & 2 after, ORF63(ICP22)—3 before & 4 after, and glycoproteins ORF37(gH)—4 before & 6 after, and ORF68(gE)—4 before & 7 after. Our findings are summarized in FIG. 9. Bars indicate reactive VZV proteins. Day 0=memory left over responses to VZV in adults that are left over from childhood chickenpox and are not related to the current FDA licensed vaccine. Day 28=responses that are a combination of leftover childhood immune memory and boosting by vaccine. ORF68=gE is good, but there are other good proteins. For example, ORF4, ORF18, ORF37, etc. These VZV ORFs are rational compositions of matter for candidate new VZV safe, protein subunit vaccines for either prevention of childhood chickenpox or prevention of adult shingles.

Throughout this application various publications are referenced. The disclosures of these publications in their entireties are hereby incorporated by reference into this application in order to describe more fully the state of the art to which this invention pertains.

Those skilled in the art will appreciate that the conceptions and specific embodiments disclosed in the foregoing description may be readily utilized as a basis for modifying or designing other embodiments for carrying out the same purposes of the present invention. Those skilled in the art will also appreciate that such equivalent embodiments do not depart from the spirit and scope of the invention as set forth in the appended claims.

1. An isolated polynucleotide encoding an alphaherpesvirus multi-epitope polypeptide, wherein the alphaherpesvirus multi-epitope polypeptide comprises a plurality of alphaherpesvirus peptide epitopes linked in a series, wherein each epitope in the series is linked to an adjacent epitope by a spacer, wherein the spacer comprises a bond, an amino acid, or a peptide comprising at least two amino acids, and wherein the plurality of alphaherpesvirus peptide epitopes comprises at least one epitope selected from Table 1.

2. The polynucleotide of claim 1, wherein the plurality of peptide epitopes comprises at least two epitopes selected from Table 1.

3. The polynucleotide of claim 1, wherein the plurality of peptide epitopes comprises the epitope ELRAREEXY, wherein X is A or S (SEQ ID NO: 58).

4. The polynucleotide of claim 1, wherein the plurality of peptide epitopes comprises the epitope QPMRLYSTCLYHPNA (SEQ ID NO: 36).

5. The polynucleotide of claim 1, wherein the plurality of peptide epitopes comprises at least one epitope identified in Table 1 as a CD4 epitope and at least one epitope identified in Table 1 as a CD8 epitope.

6. The polynucleotide of claim 1, wherein the plurality of peptide epitopes comprises at least one epitope identified as an HLA A*0201 epitope, and at least one epitope identified as an HLA A*2902 epitope.

7. The polynucleotide of claim 1, wherein the plurality of peptide epitopes comprises at least one epitope identified in Table 1 as an HSV-1 epitope, at least one epitope identified in Table 1 as an HSV-2 epitope, and at least one epitope identified in Table 1 as a VZV epitope.

8. The polynucleotide of claim 1, which further encodes a toll-like receptor (TLR) ligand or ubiquitin.

9. A vector comprising the polynucleotide of claim 1.

10. A host cell transformed with the vector of claim 9.

11. A recombinant alphaherpesvirus multi-epitope polypeptide encoded by the polynucleotide of claim 1.

12. A pharmaceutical composition comprising the polypeptide of claim 11 or a polynucleotide encoding same, and a pharmaceutically acceptable carrier.

13. The pharmaceutical composition of claim 12, further comprising an adjuvant.

14. A recombinant virus genetically modified to express the alphaherpesvirus multi-epitope polypeptide of claim 11.

15. The recombinant virus of claim 14 which is a vaccinia virus, canary pox virus or adenovirus.

16. A pharmaceutical composition comprising the recombinant virus of claim 14 and a pharmaceutically acceptable carrier.

17. A method of producing immune cells directed against alphaherpesviruses comprising contacting an immune cell with an antigen-presenting cell, wherein the antigen-presenting cell is modified to present multiple epitopes included in the recombinant polypeptide of claim 11.

18. The method of claim 17, wherein the immune cell is a T cell.

19. The method of claim 18, wherein the T cell is a CD4+ or CD8+ T cell.

20. A method of killing an alphaherpesvirus infected cell comprising contacting the infected cell with an immune cell produced by the method of claim 17.

21. A method of inhibiting HSV or VZV replication comprising contacting a HSV or VZV with an immune cell produced by the method of claim 17.

22. A method of enhancing secretion of antiviral or immunomodulatory lymphokines comprising contacting an HSV or VZV infected cell with an immune cell produced by the method of claim 17.

23. A method of enhancing production of HSV- or VZV-specific antibody comprising contacting an HSV or VZV infected cell in a subject with an immune cell produced by the method of claim 17.

24. A method of enhancing proliferation of HSV- or VZV-specific T cells comprising contacting the HSV- or VZV-specific T cells with an isolated polypeptide that comprises an epitope as recited in Table 1.

25. A method of inducing an immune response to an HSV or VZV infection in a subject comprising administering the composition of claim 12 to the subject.

26. A method of treating an HSV or VZV infection in a subject comprising administering the composition of claim 12 to the subject.

27. A method of treating an HSV or VZV infection in a subject comprising administering an antigen-presenting cell modified to present an epitope as recited in Table 1.


SEQUENCE LISTING (58 sequences)

SEQ ID NO: 1 - protein, 15 aa, Varicella zoster
STGDIIYMSPFFGLR

SEQ ID NO: 2 - protein, 15 aa, Varicella zoster
IIYMSPFFGLRDGAY

SEQ ID NO: 3 - protein, 15 aa, Varicella zoster
TRQPIGVFGTMNSQY

SEQ ID NO: 4 - protein, 15 aa, Varicella zoster
IGVFGTMNSQYSDCD

SEQ ID NO: 5 - protein, 15 aa, Varicella zoster
YGLYNSQFLALMPTV

SEQ ID NO: 6 - protein, 15 aa, Varicella zoster
NSQFLALMPTVSSAQ

SEQ ID NO: 7 - protein, 13 aa, Varicella zoster
FIFTFLSAADDLV

SEQ ID NO: 8 - protein, 13 aa, Varicella zoster
IHHYYIEQECIEV

SEQ ID NO: 9 - protein, 13 aa, Varicella zoster
SSFAAIAYLRNNG

SEQ ID NO: 10 - protein, 13 aa, Varicella zoster
NKRVFCEAVRRVA

SEQ ID NO: 11 - protein, 12 aa, Varicella zoster
PYIKIQNTGVSV

SEQ ID NO: 12 - protein, 15 aa, Varicella zoster
QPMRLYSTCLYHPNA

SEQ ID NO: 13 - protein, 15 aa, Herpes simplex virus 1
ATGDFVYMSPFYGYR

SEQ ID NO: 14 - protein, 15 aa, Herpes simplex virus 1
FVYMSPFYGYREGSH

SEQ ID NO: 15 - protein, 15 aa, Herpes simplex virus 1
ARGAIGVFGTMNSMY

SEQ ID NO: 16 - protein, 15 aa, Herpes simplex virus 1
IGVFGTMNSMYSDCD

SEQ ID NO: 17 - protein, 15 aa, Herpes simplex virus 1
HGLRNSQFVALMPTA

SEQ ID NO: 18 - protein, 15 aa, Herpes simplex virus 1
NSQFVALMPTAASAQ

SEQ ID NO: 19 - protein, 13 aa, Herpes simplex virus 1
FLFAFLSAADDLV

SEQ ID NO: 20 - protein, 13 aa, Herpes simplex virus 1
ILHYYVEQECIEV

SEQ ID NO: 21 - protein, 13 aa, Herpes simplex virus 1
ASFAAIAYLRTNN

SEQ ID NO: 22 - protein, 13 aa, Herpes simplex virus 1
NKRVFCAAVGRLA

SEQ ID NO: 23 - protein, 12 aa, Herpes simplex virus 1
PYLRIQNTGVSV

SEQ ID NO: 24 - protein, 15 aa, Herpes simplex virus 1
AEMRIYESCLYHPQL

SEQ ID NO: 25 - protein, 15 aa, Herpes simplex virus 2
ATGDFVYMSPFYGYR

SEQ ID NO: 26 - protein, 15 aa, Herpes simplex virus 2
FVYMSPFYGYREGSH

SEQ ID NO: 27 - protein, 15 aa, Herpes simplex virus 2
ARGAIGVFGTMNSAY

SEQ ID NO: 28 - protein, 15 aa, Herpes simplex virus 2
IGVFGTMNSAYSDCD

SEQ ID NO: 29 - protein, 15 aa, Herpes simplex virus 2
HGLRNSQFIALMPTA

SEQ ID NO: 30 - protein, 15 aa, Herpes simplex virus 2
NSQFIALMPTAASAQ

SEQ ID NO: 31 - protein, 13 aa, Herpes simplex virus 2
FLFAFLSAADDLV

SEQ ID NO: 32 - protein, 13 aa, Herpes simplex virus 2
ILHYYVEQECIEV

SEQ ID NO: 33 - protein, 13 aa, Herpes simplex virus 2
ASFAAIAYLRTNN

SEQ ID NO: 34 - protein, 13 aa, Herpes simplex virus 2
NKRVFCAAVGRLA

SEQ ID NO: 35 - protein, 12 aa, Herpes simplex virus 2
PYLRVQNTGVSV

SEQ ID NO: 36 - protein, 15 aa, Herpes simplex virus 2
ADMRIYEACLYHPQL

SEQ ID NO: 37 - protein, 9 aa, Varicella zoster
FLMEDQTLL

SEQ ID NO: 38 - protein, 9 aa, Varicella zoster
ILIEGIFFV

SEQ ID NO: 39 - protein, 9 aa, Varicella zoster
AVLCLYLMY

SEQ ID NO: 40 - protein, 9 aa, Varicella zoster
YMANLILKY

SEQ ID NO: 41 - protein, 13 aa, Varicella zoster
VELRAREEAYTKL

SEQ ID NO: 42 - protein, 9 aa, Varicella zoster
ELRAREEAY

SEQ ID NO: 43 - protein, 9 aa, Herpes simplex virus 1
FLWEDQTLL

SEQ ID NO: 44 - protein, 9 aa, Herpes simplex virus 1
ILIEGIFFA

SEQ ID NO: 45 - protein, 9 aa, Herpes simplex virus 1
AVLCLYLLY

SEQ ID NO: 46 - protein, 9 aa, Herpes simplex virus 1
YMANQILRY

SEQ ID NO: 47 - protein, 13 aa, Herpes simplex virus 1
AELRAREESYRTV

SEQ ID NO: 48 - protein, 9 aa, Herpes simplex virus 1
ELRAREESY

SEQ ID NO: 49 - protein, 9 aa, Herpes simplex virus 2
FLWEDQTLL

SEQ ID NO: 50 - protein, 9 aa, Herpes simplex virus 2
ILIEGVFFA

SEQ ID NO: 51 - protein, 9 aa, Herpes simplex virus 2
AVLCLYLLY

SEQ ID NO: 52 - protein, 9 aa, Herpes simplex virus 2
YMANQILRY

SEQ ID NO: 53 - protein, 13 aa, Herpes simplex virus 2
GELRAREESYRTV

SEQ ID NO: 54 - protein, 9 aa, Herpes simplex virus 2
ELRAREESY

SEQ ID NO: 55 - protein, 410 aa, Varicella zoster
MECNLGTEHPSTDTWNRSKTEQAVVDAFDESLFGDVASDIGFETSLYSHAVKTAPSPPWVASPKILYQQLIRDLDFSEGPRLLSCLETWNEDLFSCFPINEDLYSDMMVLSPDPDDVISTVSTKDHVEMFNLTTRGSVRLPSPPKQPTGLPAYVQEVQDSFTVELRAREEAYTKLLVTYCKSIIRYLQGTAKRTTIGLNIQNPDQKAYTQLRQSILLRYYREVASLARLLYLHLYLTVTREFSWRLYASQSAHPDVFAALKFTWTERRQFTCAFHPVLCNHGIVLLEGKPLTASALREINYRRRELGLPLVRCGLVEENKSPLVQQPSFSVHLPRSVGFLTHHIKRKLDAYAVKHPQEPRHVRADHPYAKVVENRNYGSSIEAMILAPPSPSEILPGDPPRPPTCGFLTR

SEQ ID NO: 56 - protein, 490 aa, Herpes simplex virus 1
MDLLVDELFADMNADGASPPPPRPAGGPKNTPAAPPLYATGRLSQAQLMPSPPMPVPPAALFNRLLDDLGFSAGPALCTMLDTWNEDLFSALPTNADLYRECKFLSTLPSDVVEWGDAYVPERTQIDIRAHGDVAFPTLPATRDGLGLYYEALSRFFHAELRAREESYRTVLANFCSALYRYLRASVRQLHRQAHMRGRDRDLGEMLRATIADRYYRETARLARVLFLHLYLFLTREILWAAYAEQMMRPDLFDCLCCDLESWRQLAGLFQPFMFVNGALTVRGVPIEARRLRELNHIREHLNLPLVRSAATEEPGAPLTTPPTLHGNQARASGYFMVLIRAKLDSYSSFTTSPSEAVMREHAYSRARTKNNYGSTIEGLLDLPDDDAPEEAGLAAPRLSFLPAGHTRRLSTAPPTDVSLGDELHLDGEDVAMAHADALDDFDLDMLGDGDSPGPGFTPHDSAPYGALDMADFEFEQMFTDALGIDEYGG

SEQ ID NO: 57 - protein, 490 aa, Herpes simplex virus 2
MDLLVDDLFADADGVSPPPPRPAGGPKNTPAAPPLYATGRLSQAQLMPSPPMPVPPAALFNRLLDDLGFSAGPALCTMLDTWNEDLFSGFPTNADMYRECKFLSTLPSDVIDWGDAHVPERSPIDIRAHGDVAFPTLPATRDELPSYYEAMAQFFRGELRAREESYRTVLANFCSALYRYLRASVRQLHRQAHMRGRNRDLREMLRTTIADRYYRETARLARVLFLHLYLFLSREILWAAYAEQMMRPDLFDGLCCDLESWRQLACLFQPLMFINGSLTVRGVPVEARRLRELNHIREHLNLPLVRSAAAEEPGAPLTTPPVLQGNQARSSGYFMLLIRAKLDSYSSVATSEGESVMREHAYSRGRTRNNYGSTIEGLLDLPDDDDAPAEAGLVAPRMSFLSAGQRPRRLSTTAPITDVSLGDELRLDGEEVDMTPADALDDFDLEMLGDVESPSPGMTHDPVSYGALDVDDFEFEQMFTDAMGIDDFGG

SEQ ID NO: 58 - protein, 9 aa, Alphaherpesvirus
  MISC_FEATURE (8)..(8): Xaa is A or S
ELRAREEXY